(19) United States
(12) Patent Application Publication
(10) Pub. No.: US 2026/0265708 A1
(43) Pub. Date: Sep. 10, 2026

(54) NEW FUCOSYLTRANSFERASES FOR IN VIVO SYNTHESIS OF COMPLEX FUCOSYLATED HUMAN MILK OLIGOSACCHARIDES

(71) Applicant: DSM IP Assets B.V., TE HEERLEN (NL)

(72) Inventor: Manos PAPADAKIS, Hørsholm (DK)

(21) Appl. No.: 18/994,173

(22) PCT Filed: Jul. 13, 2023
(86) PCT No.: PCT/EP2023/069573

§ 371 Date: Jan. 14, 2025

(30) Foreign Application Priority Data
Jul. 15, 2022 (DK) PA202200688
Dec. 22, 2022 (DK) PA202201203

(51) Int. Cl. C12N 9/10; C12P 19/04; C12P 19/18

(52) U.S. Cl. CPC C12N 9/1051; C12P 19/04; C12P 19/18

(57) ABSTRACT

The present invention relates to the production of fucosylated Human Milk Oligosaccharides (HMOs), and in particular to the production of complex fucosylated HMOs with five or more monosaccharide units, such as LNFP-V and LNDFH-II, from precursor oligosaccharides and the genetic engineering cells and α-1,3(4)-fucosyltransferases suitable for use in said production of suitable for use in said production, as well as to methods for producing said fucosylated HMOs.

### CROSS-REFERENCE TO RELATED APPLICATIONS

This application is a national stage entry pursuant to 35 U.S.C. § 371 of International Application No. PCT/EP2023/069573, filed on Jul. 13, 2023, which claims priority to Denmark Application Nos. PA202201203, filed on Dec. 22, 2022 and PA202200688, filed on Jul. 15, 2022, the entire contents of all of which are hereby incorporated by reference in their entirety.

### SEQUENCE LISTING

The computer-readable Sequence Listing submitted on Nov. 5, 2025 and identified as follows: 57,940 bytes ST.26 XML document file named “Sequence_Listing.xml,” created Nov. 4, 2025, is incorporated herein by reference in its entirety.

### FIELD

The present invention relates to the production of complex fucosylated Human Milk Oligosaccharides (HMOs) and in particular to the production of complex fucosylated HMOs with five or more monosaccharide units, such as LNFP-V and LNDFH-II, as well as to genetically engineered cells suitable for use in said production.

### BACKGROUND

The design and construction of bacterial cell factories to produce fucosylated Human Milk Oligosaccharides (HMOs), especially for producing more complex fucosylated Human Milk Oligosaccharides (HMOs), is of paramount importance to provide innovative and scalable solutions for the more complex products of tomorrow.

Production of complex di-fucosylated HMOs either needs a multiple assortment of enzymes, or multi-specific enzymes, such as bi-specific glycosyltransferases capable of producing complex di-fucosylated HMOs. Multi-specific enzymes are preferred due to the lower genetical burden of introducing them to the host cell.

It has e.g., been suggested in WO2020/115671, that the α1,3-fucosyltransferase, FutA and variants thereof, can produce the complex HMO LNFP-V, and in WO2019/008133 it has been described that the α1,3-fucosyltransferase FucT109 appears to fucosylate both the glucose (Glc) and N-acetylglucosamine (GlcNAc) moiety of Lacto-N-neotetraose (LNnT), thus potentially generating a mixture containing the complex fucosylated HMOs LNFP-III and LNFP-VI, but FucT109 only appear to fucosylate the Glc moiety in LNT.

Dumon et al., 2004 (Biotechnol. Prog. 2004, 20, 412-419) further describes that the α1,3-fucosyltransferase, FutA, produces a mixture comprising the complex fucosylated HMOs LNDFH-III (LNnDFH-II) and LNFP-VI (LNnFP-V). Dumon et. al., 2004 does not disclose di-fucosylation with LNT backbone.

Baumgartner et al 2015 (Bioorganic & Medicinal Chemistry 23:6799-6806) disclose that the alpha-1,4-fucosyltransferase FucTIII can produce LNDFH-II, but is silent with respect to LNFP-V formation.

In summary, production of fucosylated HMOs, especially of more complex fucosylated HMOs, is challenging due to the lack of fucosyltransferases with the desired substrate specificity, as well as low production yields of the desired fucosylated HMOs as compared to other HMO products present after fermentation, such as HMO precursor products, which may require laborious separation procedures.

### SUMMARY

The need for bi-specific glycosyltransferases for the production of complex di-fucosylated HMOs with an LNT backbone, and in particular for the production of LNDFH-II, is in the present invention solved by the identification of a selection of α-1,3(4)-fucosyltransferases which exhibit low or no specificity for the galactose moiety in LNT as a substrate (acceptor oligosaccharide) for fucosylation reactions, but which are highly substrate specific for the N-acetylglucosamine (GlcNAc) and glucose (Glu) moieties in LNT, thus producing the complex fucosylated HMO LNDFH-II, or mixtures of HMOs that comprise LNDFH-II, and which have a high total content of fucosylated HMOs. The α-1,3(4)-fucosyltransferases presented herein are therefore useful in the production of LNDFH-II, as well as mixtures of LNDFH-II and LNFP-V. Hence, provided herein are enzymes, mixtures, compositions, uses, genetically engineered cells and methods for the production of LNDFH-II or mixtures of HMOs that comprise LNDFH-II, and which have a high total content of fucosylated HMOs.

In a first aspect, the invention relates to a genetically engineered cell, which is capable of producing LNDFH-II, comprising a recombinant nucleic acid sequence encoding a glycosyltransferase is selected from the group consisting of,
a. Bgall1 comprising or consisting of an amino acid sequence according to SEQ ID NO: 1, or a functional homologue thereof with an amino acid sequence that is at least 80% identical to SEQ ID NO: 1,
b. Murba1 comprising or consisting of an amino acid sequence according to SEQ ID NO: 2, or a functional homologue thereof with an amino acid sequence that is at least 80% identical to SEQ ID NO: 2 and
c. Bbac1 comprising or consisting of an amino acid sequence according to SEQ ID NO: 3, or a functional homologue thereof with an amino acid sequence that is at least 80% identical to SEQ ID NO: 3, and
d. Para1 comprising or consisting of an amino acid sequence according to SEQ ID NO: 4, or a functional homologue thereof with an amino acid sequence that is at least 80% identical to SEQ ID NO: 4.

Where the initial substrate is lactose it is preferred that the genetically engineered cell further comprises one or more recombinant nucleic acid sequence(s) encoding a β-1,3-N-acetylglucosaminyltransferase and a β-1,3-galactosyltransferase.

In a second aspect, the invention relates to a method for producing one or more fucosylated HMOs, preferably LNDFH-II and/or mixtures of HMOs comprising LNDFH-II, said method comprising culturing a genetically engineered cell capable of producing LNDFH-II, comprising a recombinant nucleic acid sequence encoding a fucosyltransferase with α-1,3-fucosyltransferase and α-1,4-fucosyltransferase activity (also termed a α-1,3(4)-fucosyltransferase), which is capable of fucosylating an acceptor oligosaccharide at a GlcNAc moiety and a Glc moiety, wherein the glycosyltransferase is selected from the group consisting of Bgall1 comprising or consisting of an amino acid sequence according to SEQ ID NO: 1, or a functional homologue thereof with an amino acid sequence that is at least 80% identical to SEQ ID NO: 1, Murba1 comprising or consisting of an amino acid sequence according to SEQ ID NO: 2, or a functional homologue thereof with an amino acid sequence that is at least 80% identical to SEQ ID NO: 2 and Bbac1 comprising or consisting of an amino acid sequence according to SEQ ID NO: 3, or a functional homologue thereof with an amino acid sequence that is at least 80% identical to SEQ ID NO: 3. Preferably, the cell further comprises one or more recombinant nucleic acid sequence(s) encoding a β-1,3-N-acetylglucosaminyltransferase and a β-1,3-galactosyltransferase.

In a third aspect, the invention relates to the use of an enzyme with α-1,3-fucosyltransferase and α-1,4-fucosyltransferase activity, which is capable of fucosylating an acceptor oligosaccharide at a GlcNAc moiety and a Glc moiety for the production of one or more fucosylated HMOs, preferably LNDFH-II and mixtures of HMOs comprising LNDFH-II.

In a fourth aspect, the invention relates to a mixture of HMOs, produced by a method of the current invention, comprising or consisting essentially of
a) LNDFH-II, LNFP-V and LNT, or
b) LNDFH-II, LNFP-V and 3FL, or
c) LNDFH-II, LNFP-V, LNT and 3FL, or
d) LNDFH-II, LNT-II, LNFP-V, LNFP-II, and LNT

The invention further relates to compositions comprising the mixtures of a)-d) and use of said compositions in an infant formula, a dietary supplement, or medical nutrition.

### BRIEF DESCRIPTION OF FIGURES

FIG. 1. Overview of the synthesis of complex fucosylated HMO with an LNT-backbone.

### DETAILED DESCRIPTION

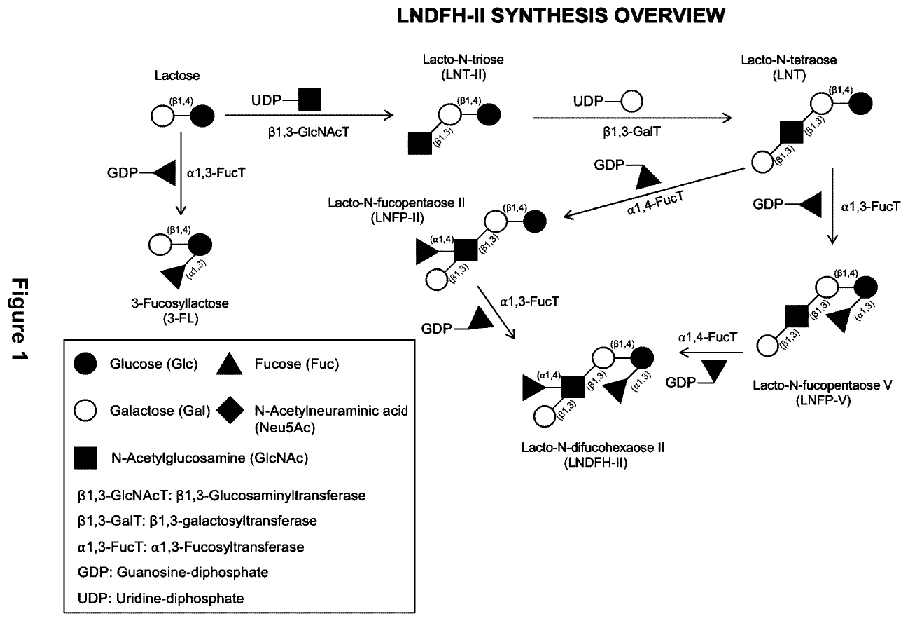

The present invention approaches the biotechnological challenges of in vivo HMO production of, in particular, complex fucosylated HMOs with an LNT-backbone, which comprise at least five monosaccharide units, of which at least one monosaccharide unit is a fucosyl unit, such as in particular the di-fucosylated complex HMOs with at least six monosaccharide units. Examples of such complex fucosylated HMOs are LNFP-V and LNDFH-II. The present invention offers specific strain engineering solutions for producing specific complex fucosylated HMOs, in particular, LNFP-V and/or LNDFH-II, by exploiting the dual substrate specificity of the α-1,3(4)-fucosyltransferases disclosed herein towards the N-acetylglucosamine (GlcNAc) and Glucose (Glc) moieties in LNT (see FIG. 1).

The complex fucosylated HMOs with the LNT backbone can be produced from lactose as the initial substrate, but in so far, that the cell is capable of internalizing for example LNT-II or LNT (see for example WO2023/099680), these may also serve as initial substrates.

Production of LNFP-V and/or LNDFH-II

The advantage of using the α-1,3(4)-fucosyltransferases of the present disclosure is their ability to specifically recognize and fucosylate both the GlcNAc and Glucose moieties in LNT, to generate LNFP-II and/or LNFP-V, which is in turn further fucosylated at the GlcNAc or Glucose moiety to produce the di-fucosylated HMO, LNDFH-II, (see FIG. 1). In essence, the present disclosure describes several newly identified enzymes with dual α-1,3-fucosyltransferase and 1,4-fucosyltransferase activity (α-1,3(4)-fucosyltransferase) that are capable of producing the complex fucosylated HMOs LNFP-V and/or LNDFH-II. Thus, where LNT is available in sufficient amounts inside the genetically engineered cell to saturate the enzymes, very little, if any LNFP-II, is produced by the preferred α-1,3(4)-fucosyltransferase described in the present disclosure.

Only one of the α-1,3(4)-fucosyltransferases, Para1, described herein produce similar levels of LNFP-II and LNFP-V when expressed in the cell. From example 1, it can be seen that when the expression of Para1 is increased, more LNDFH-II is produced indicating that higher amounts of the expressed enzyme is beneficial to achieve the di-fucosylation.

Without being bound by theory, the four other α-1,3(4)-fucosyltransferases described herein, Bgall1, Murba1, Bbac1 and FutA all appear to generate LNDFH-II via LNFP-V as the preferred intermediate (see FIG. 1), since little or no LNFP-II is produced. The traits of these α-1,3(4)-fucosyltransferases are therefore well-suited for high-level industrial production of LNDFH-II and/or LNFP-V, or mixtures thereof, without production of high levels of alternatively complex fucosylated HMOs (side products), such as LNFP-II and/or other by-product HMOs, such as 3FL, LNT-II and/or LNnT. pLNH2 is also a potential oligosaccharide by-product, it is not officially reported as an HMO although it may very well be present in human mothers milk in small amounts.

Furthermore, the α-1,3(4)-fucosyltransferases described herein are also well-suited for producing LNDFH-II and mixtures comprising LNDFH-II, LNFP-V, LNT and/or 3FL.

The genetically engineered cells of the present disclosure, which express any one or more of the α-1,3(4)-fucosyltransferases disclosed herein, with high substrate specificity for the GlcNAc and Glucose moieties in LNT, for the first time enable the production of titers of LNDFH-II which exceed 5%, such as exceeds 10%, such as exceeds 15%, such as such as exceeds 20% of the total amount of HMO produced. In some instances two or more copies of the nucleic acid encoding the α-1,3(4)-fucosyltransferases is needed to produce more than 5% of LNDFH-II. From the mixtures of HMOs produced by the genetically engineered cells LNDFH-II as well as LNFP-V can potentially be purified.

Thereby, the present disclosure enables a more efficient biotechnological production of more complex fucosylated HMOs, such as LNFP-V and LNDFH-II, either in purified form or as mixtures with the fucosylated HMOs being the most predominant, e.g., exceeding 60% of the total HMOs produced.

In the following sections, individual elements of the invention, and in particular of the genetically engineered cell are described. It is understood that these elements can be combined across the individual sections.

### Oligosaccharides

In the present context, the term “oligosaccharide” means a sugar polymer containing at least three monosaccharide units, i.e., a tri-, tetra-, penta-, hexa- or higher oligosaccharide. The oligosaccharide can have a linear or branched structure containing monosaccharide units that are linked to each other by interglycosidic linkages. Particularly, the oligosaccharide comprises a lactose residue at the reducing end and one or more naturally occurring monosaccharides of 5-9 carbon atoms selected from aldoses (e.g., glucose, galactose, ribose, arabinose, xylose, etc.), ketoses (e.g., fructose, sorbose, tagatose, etc.), deoxysugars (e.g. rhamnose, fucose, etc.), deoxy-aminosugars (e.g. N-acetyl-glucosamine, N-acetyl-mannosamine, N-acetyl-galactosamine, etc.), uronic acids and ketoaldonic acids (e.g. N-acetylneuraminic acid). Preferably, the oligosaccharide is an HMO.

### Human Milk Oligosaccharide (HMO)

Preferred oligosaccharides of the disclosure are human milk oligosaccharides (HMOs).

The term “human milk oligosaccharide” or “HMO” in the present context means a complex carbohydrate found in human breast milk. The HMOs have a core structure comprising a lactose unit at the reducing end that can be elongated by one or more β-N-acetyl-lactosaminyl and/or one or more β-lacto-N-biosyl unit, and this core structure can be substituted by an α-L-fucopyranosyl and/or an α-N-acetyl-neuraminyl (fucosyl) moiety. HMO structures are e.g., disclosed by Xi Chen in Chapter 4 of Advances in Carbohydrate Chemistry and Biochemistry 2015 vol 72.

The present invention focuses on fucosylated HMO's. Examples of fucosylated HMOs include, 2′-fucosyllactose (2′FL), lacto-N-fucopentaose I (LNFP-I), lacto-N-difucohexaose I (LNDFH-I), 3-fucosyllactose (3FL), difucosyllactose (DFL), lacto-N-fucopentaose II (LNFP-II), lacto-N-fucopentaose III (LNFP-III), lacto-N-difucohexaose III (LNDFH-III), fucosyl-lacto-N-hexaose II (FLNH-II), lacto-N-fucopentaose V (LNFP-V), lacto-N-fucopentaose VI (LNFP-VI), lacto-N-difucohexaose II (LNDFH-II), fucosyl-lacto-N-hexaose I (FLNH-I), fucosyl-para-lacto-N-hexaose I (FpLNH-I), fucosyl-para-lacto-N-neohexaose II (F-pLNnH II), fucosyl-lacto-N-neohexaose (FLNnH), 3-fucosyl-3′-fucosyllactose (FSL), fucosyl-LST-a (FLST-a), fucosyl-LST b (FLST b), fucosyl-LST-c (FLST-c), fucosyl-LST d (FLST-d) and fucosyl-lacto-N-hexaose (SLNH).

In the context described herein, complex fucosylated HMOs are fucosylated HMOs that comprises at least 5 monosaccharide units of which at least one monosaccharide unit is a fucosyl unit, non-limiting examples of complex fucosylated HMOs are the fucosylated HMOs consisting of 5 monosaccharide units e.g., LNFP-I, LNFP-II, LNFP-III, LNFP-V and LNFP-VI and complex fucosylated HMO with 6 monosaccharide units such as but not limited to the di-fucosylated HMOs LNDFH-I, LNDFH-II and LNDFH-III or the sialyl-fucosyl HMOs FLST-a, FLST-b, FLST-c and FLST-d. Preferably, a complex fucosylated HMO is one that requires at least three different glycosyltransferase activities to be produced from lactose as the initial substrate, e.g., the formation of LNFP-V requires an α-1,3-fucosyltransferase, a β-1,3-N-acetyl-glucosaminyl-transferase and a β-1,3-galactosyltransferase (see FIG. 1), the formation of LNFP-II requires an α-1,4-fucosyltransferase, a β-1,3-N-acetyl-glucosaminyl-transferase and α-1,3-galactosyltransferase (see FIG. 1), and the formation of LNDFH-II requires an α-1,3-fucosyltransferase, an α-1,4-fucosyltransferase, a β-1,3-N-acetyl-glucosaminyl-transferase and a β-1,3-galactosyltransferase, wherein the α-1,3-fucosyltransferase and α-1,4-fucosyltransferase, may be a single fucosyltransferase with dual activity, also termed a α-1,3(4)-fucosyltransferase.

In particular, the fucosylated HMO(s) produced is/are selected from complex fucosylated HMOs comprising at least five monosaccharide units of which at least one monosaccharide unit is a fucosyl unit. Moreover, in embodiments, the fucosylated HMOs is/are selected from complex fucosylated HMOs with an LNT backbone structure, preferably, selected from the group consisting of LNFP-II, LNFP-V and LNDFH-II. Examples of fucosylated HMOs with an LNT backbone structure are lacto-N-fucopentaose I (LNFP-I), lacto-N-fucopentaose II (LNFP-II), lacto-N-fucopentaose V (LNFP-V), Lacto-N-difucohexaose I (LNDFH-I), Lacto-N-difucohexaose II (LNDFH-II), sialyl-lacto-N-fucopentaose I (S-LNFP-I or FLST-b), sialyl-lacto-N-fucopentaose II (S-LNFP-II or FLST-a), Mono-Fucosyl-lacto-N-hexaose I (F-LNH-I), Mono-Fucosyl-lacto-N-hexaose II (F-LNH-II), Mono-fucosyl-lacto-N-hexaose III (F-LNH-III), Difucosyl-lacto-N-hexaose I (DF-LNH-I), Difucosyl-lacto-N-hexaose II (DF-LNH-II), Difucosyl-Lacto-N-hexaose III (DF-LNH-III), Trifucosyl-lacto-N-hexaose (TF-LNH), Fucosyl-sialyl-lacto-N-hexaose I (FS-LNH) and Disialyl-fucosyl-lacto-N-hexaose II (DS-F-LNH-II).

In embodiments of the present disclosure, the fucosyltransferases described herein predominantly fucosylates both the N-acetylglucosamine (GlcNAc) and Glucose (Glc) moieties of LNT while also being capable of fucosylating the Glucose (Glc) moiety of lactose. In that regard, the one or more fucosylated HMOs is preferably 3FL, LNFP-V, LNFP-II and LNDFH-II, or 3FL, LNFP-V, and LNDFH-II.

In other embodiments, the fucosyltransferases disclosed herein only fucosylates the N-acetylglucosamine (GlcNAc) and/or Glucose (Glc) moiety of LNT. In that regard, the one or more fucosylated HMOs is preferably LNDFH-II, LNFP-II and LNFP-V or LNDFH-II and LNFP-V.

In human milk, about 60% of the content of HMOs are fucosylated HMOs, thus production of mixtures comprising a high content of fucosylated HMOs is highly desirable for the production of more natural mixtures of HMOs. In embodiments of the present disclosure at least 80 molar %, such as at least 85 molar %, 86 molar %, 87 molar %, 88 molar %, 89 molar %, 90 molar %, 91 molar %, 92 molar %, 93 molar %, 94 molar %, 95 molar %, 96 molar %, 97 molar % or at least 98 molar %, of the produced HMOs are fucosylated HMOs.

### An Acceptor Oligosaccharide

A genetically engineered cell described herein comprises a recombinant nucleic acid sequence encoding a fucosyltransferase with α-1,3(4)-fucosyltransferase activity capable of transferring fucose from an activated sugar to the GlcNAc and/or Glc moiety of an acceptor oligosaccharide, in an α-1,4 linkage on GlcNAc moiety or α-1,3 linkage on the Glc moiety.

In the context described herein, an acceptor oligosaccharide is an oligosaccharide that can act as a substrate for a glycosyltransferase capable of transferring a glycosyl moiety from a glycosyl donor to the acceptor oligosaccharide. The glycosyl donor is preferably a nucleotide-activated sugar as described in the section on “Glycosyl-donor-nucleotide-activated sugar pathways”. Preferably, the acceptor oligosaccharide is a precursor for making a more complex HMO and can also be termed the precursor molecule.

The acceptor oligosaccharide can be either an intermediate product of the present fermentation process, an end-product of a separate fermentation process employing a separate genetically engineered cell, or an enzymatically or chemically produced molecule.

In the present context, said acceptor oligosaccharide for the α-1,3(4)-fucosyltransferase is preferably lacto-N-tetraose (LNT), which is produced from the precursor molecule lacto-N-triose II (LNT-II) (e.g., acceptor for the β-1,3-galactosyltransferase), which is produced from the initial precursor molecule lactose (e.g., acceptor for the β-1,3-N-acetyl-glucosaminyl-transferase) (see FIG. 1). In addition the acceptor oligosaccharide for the α-1,3(4)-fucosyltransferase may also be lacto-N-fucopentaose II (LNFP-II) which is produced from the precursor molecule LNT (e.g., acceptor for the α-1,4-fucosyltransferase) or the lacto-N-fucopentaose V (LNFP-V) which is produced from the precursor molecule LNT (e.g., acceptor for the α-1,3-fucosyltransferase). The initial precursor molecule is preferably fed to the genetically engineered cell, which is capable of producing e.g., LNT-II, LNT, LNFP-II, LNFP-V and/or LNDFH-II from the precursor. Most often the initial precursor is lactose, and the genetically engineered cell is capable of producing the intermediate precursors (acceptor oligosaccharides, e.g. LNT-II and LNT) inside the cell. The initial precursor may however also be LNT-II or LNT if the cell is capable of importing at least one of these compounds.

### Glycosyltransferases

The genetically engineered cell described herein comprises at least one recombinant nucleic acid sequence encoding at least one glycosyltransferase, i.e., a fucosyltransferase, capable of transferring a fucosyl residue from a fucosyl donor to an acceptor oligosaccharide to synthesize one or more fucosylated human milk oligosaccharide products.

The genetically engineered cell described herein may comprise one or more further recombinant nucleic acids encoding one or more recombinant and/or heterologous glycosyltransferases capable of transferring a glycosyl residue from a glycosyl donor to an acceptor oligosaccharide. Preferably, the additional glycosyltransferase(s) enables the genetically engineered cell to synthesize LNT from a precursor molecule, such as lactose or LNT-II. In embodiments, the genetically engineered cell described herein, comprises one or more further recombinant nucleic acid encoding one or more recombinant and/or heterologous glycosyltransferase.

The additional glycosyltransferase is preferably selected from the group consisting of, galactosyltransferases, glucosaminyltransferases, fucosyltransferases and N-acetylglucosaminyl transferases.

The fucosyltransferase in the genetically engineered cell described herein is an α-1,3(4)-fucosyltransferase. Preferably, the α-1,3(4)-fucosyltransferase is capable of transferring a fucose unit onto the GlcNAc and/or Glc moiety of an LNT, LNFP-II and/or LNFP-V.

In the present disclosure, the functional enzyme (α-1,3(4)-fucosyltransferase) capable of transferring a fucosyl moiety from a fucosyl donor to an acceptor oligosaccharide is selected from the group consisting of a) Bgall1 comprising or consisting of an amino acid sequence according to SEQ ID NO: 1, or a functional homologue thereof with an amino acid sequence that is at least 80% identical to SEQ ID NO: 1, b) Murba1 comprising or consisting of an amino acid sequence according to SEQ ID NO: 2, or a functional homologue thereof with an amino acid sequence that is at least 80% identical to SEQ ID NO: 2, c) Bbac1 comprising or consisting of an amino acid sequence according to SEQ ID NO: 3, or a functional homologue thereof with an amino acid sequence that is at least 80% identical to SEQ ID NO: 3. These enzymes can e.g., be used to produce LNFP-II, LNFP-V and/or LNDFH-II, d) Para1 comprising or consisting of an amino acid sequence according to SEQ ID NO: 4, or a functional homologue thereof with an amino acid sequence that is at least 80% identical to SEQ ID NO: 4, and e) FutA comprising or consisting of an amino acid sequence according to SEQ ID NO: 5, or a functional homologue thereof with an amino acid sequence that is at least 80% identical to SEQ ID NO: 5.

In preferred embodiments the functional enzyme (α-1,3(4)-fucosyltransferase) capable of transferring a fucosyl moiety from a fucosyl donor to an acceptor oligosaccharide is selected from the group consisting of a) Bgall1 comprising or consisting of an amino acid sequence according to SEQ ID NO: 1, or a functional homologue thereof with an amino acid sequence that is at least 80% identical to SEQ ID NO: 1, b) Murba1 comprising or consisting of an amino acid sequence according to SEQ ID NO: 2, or a functional homologue thereof with an amino acid sequence that is at least 80% identical to SEQ ID NO: 2, c) Bbac1 comprising or consisting of an amino acid sequence according to SEQ ID NO: 3, or a functional homologue thereof with an amino acid sequence that is at least 80% identical to SEQ ID NO: 3. These enzymes can e.g., be used to produce LNFP-II, LNFP-V and/or LNDFH-II.

The preference of most of the α-1,3(4)-fucosyltransferases of the present disclosure leans towards production of a mixture of LNFP-V and LNDFH-II with only minor or no production of LNFP-II, suggesting that these enzymes have higher specificity for the Glc moiety in LNT, and the fucosylation of the GlcNAc moiety primarily occurs on LNFP-V and not LNT.

Without being bound by theory, an α-1,3(4)-fucosyltransferase that predominantly produces mixtures of LNFP-V and LNDFH-II, with little or no LNFP-II, e.g., Bgall1, Murba1, Bbac1 and FutA, is advantageous if it is desired to produce less or no alternatively complex fucosylated HMOs, such as LNFP-II, when the initial substrate is lactose. A reduced number of alternative complex fucosylated HMOs would in turn result in an easier purification of the produced HMOs, as the purification of LNDFH-II and/or LNFP-V from a mixture of HMOs predominantly comprising the intended product would be simpler, as it is easier to separate the complex fucosylated HMOs from smaller HMOs than separating different fucosylated HMOs of the same or similar size from each other, e.g., LNDFH-II from LNFP-V, or LNDFH-II from LNFP-II. Hence, the presence of only on HMO comprising five monosaccharide units, e.g., only LNFP-II or only LNFP-V in a LNDFH-II production is considered beneficial for the purification of LNDFH-II.

In preferred embodiments, the use of an α-1,3(4)-fucosyltransferase described herein results in that at least 75 molar %, such as at least 80 molar %, 85 molar %, 90 molar %, 95 molar %, 96 molar %, 97 molar % or such as at least 98 molar % of the molar content of the total HMOs produced by a cell described herein is fucosylated HMOs.

In further embodiments, the use of an α-1,3(4)-fucosyltransferase described herein results in that at least 40 molar %, such as at least 50 molar %, 60 molar %, 70 molar %, 80 molar % or such as at least 90 molar %, or such as between 40 molar % and 90 molar % or such as between 50 molar % and 80 molar % of the molar content of the total HMOs produced by a cell described herein is a mixture of LNDFH-II and LNFP-V.

In other preferred embodiments, the use of an α-1,3(4)-fucosyltransferase described herein results in that at least 1%, such as at least 5 molar %, 6 molar %, 7 molar %, 8 molar %, 9 molar %, 10 molar %, 15 molar %, 20 molar, 25 molar %, 30 molar % or such as at least 35 molar % of the molar content of the total HMOs produced by a cell described herein is LNDFH-II.

In other preferred embodiments, the use of an α-1,3(4)-fucosyltransferase described herein results in that at least 5% of the molar content of the total HMOs produced by a cell described herein is LNDFH-II.

In one embodiment, the α-1,3(4)-fucosyltransferase is Bgall1, comprising or consisting of an amino acid sequence according to SEQ ID NO: 1 or a functional homologue thereof with an amino acid sequence that is at least 80% identical to SEQ ID NO: 1. Preferably, when expressed in a suitable genetically engineered cell, Bgall1 is capable of producing at least 10%, such as at least 15% of the molar content of the total HMOs produced by a cell described herein is LNDFH-II.

In one embodiment, the α-1,3(4)-fucosyltransferase is Murba1, comprising or consisting of an amino acid sequence according to SEQ ID NO: 2 or a functional homologue thereof with an amino acid sequence that is at least 80% identical to SEQ ID NO: 2. Preferably, when expressed in sufficient amounts in a suitable genetically engineered cell, Murba1 is capable of producing at least 1%, such as at least 4% of the molar content of the total HMOs produced by a cell described herein is LNDFH-II.

In one embodiment, the α-1,3(4)-fucosyltransferase is Bbac1, comprising or consisting of an amino acid sequence according to SEQ ID NO: 3 or a functional homologue thereof with an amino acid sequence that is at least 80% identical to SEQ ID NO: 3. Preferably, when expressed in sufficient amounts in a suitable genetically engineered cell, Bbac1 is capable of producing at least 5% of the molar content of the total HMOs produced by a cell described herein is LNDFH-II.

In one embodiment, the α-1,3(4)-fucosyltransferase is Para1, comprising or consisting of an amino acid sequence according to SEQ ID NO: 4 or a functional homologue thereof with an amino acid sequence that is at least 80% identical to SEQ ID NO: 4. Preferably, when expressed in sufficient amounts in a suitable genetically engineered cell, Para1 is capable of producing at least 1%, such as at least 5% of the molar content of the total HMOs produced by a cell described herein is LNDFH-II.

In one embodiment, the α-1,3(4)-fucosyltransferase is FutA, comprising or consisting of an amino acid sequence according to SEQ ID NO: 5 or a functional homologue thereof with an amino acid sequence that is at least 80% identical to SEQ ID NO: 5. Preferably, when expressed in sufficient amounts in a suitable genetically engineered cell, FutA is capable of producing at least 5% of the molar content of the total HMOs produced by a cell described herein is LNDFH-II.

In embodiments, the expression of an α-1,3(4)-fucosyltransferase described herein in a genetically engineered cell is further combined with expression of one or more further recombinant nucleic acids encoding one or more recombinant and/or heterologous glycosyltransferases.

In embodiments described herein the cell further comprises one or more recombinant nucleic acid sequence encoding a β-1,3-galactosyltransferase.

In preferred embodiments, the expression of an α-1,3(4)-fucosyltransferase described herein, in a genetically engineered cell is combined with expression of a β-1,3-galactosyltransferase such as galTK from Helicobacter pylori. In a further embodiment, a third enzyme is expressed, such as a β-1,3-N-acetyl-glucosaminyl-transferase, e.g., LgtA from Neisseria meningitidis.

Exemplified further glycosyltransferases in addition to the α-1,3(4)-fucosyltransferases, Bgall1, Bbac1, Para1, Murba1 and FutA, are preferably selected from the glycosyltransferases described below (tables 1, 2 and 3).

### α-1,3(4)-Fucosyltransferase

The term “α-1,3(4)-fucosyltransferase” refers to a glycosyltransferase that catalyzes the transfer of fucosyl from a donor substrate, such as GDP-fucose, to an acceptor molecule in an α-1,3-linkage or α-1,4-linkage (see FIG. 1). Preferably, an α-1,3(4)-fucosyltransferase used in the present invention does not originate in the species of the genetically engineered cell, i.e., the gene encoding the α-1,3(4)-fucosyltransferase is of heterologous origin and is selected from an α-1,3(4)-fucosyltransferase identified in table 1. In the context described herein, the acceptor molecule for the α-1,3(4)-fucosyltransferase is preferably an acceptor oligosaccharide of at least four monosaccharide units with at least one glucose and at least on GlcNAc moiety, e.g., LNT. Heterologous α-1,3(4)-fucosyltransferases that are capable of transferring a fucosyl moiety onto the glucose of moiety of LNT are known in the art, specifically FutA with an amino acid sequence as provided in SEQ ID NO: 5 and encoded by the nucleic acid sequence of SEQ ID NO: 10, is known to produce LNFP-V (WO2020115671).

The fucosyltransferase can be selected from an amino acid sequence with at least 80%, such as at least 85%, such as at least 90%, such as at least 95%, or such as at least 99% sequence identity to the amino acid sequence of any one of the α-1,3(4)-fucosyltransferases listed in table 1.

TABLE 1
List of α-1,3(4)-fucosyltransferase
enzymes capable of producing LNDFH-II.

| Enzyme | | SEQ | |
|---|---|---|---|
| Name | GenBank ID¹ | ID NO: | Origin |
| Bgall1 | WP_204430034.1 | 1 | Bacteroides gallinaceum |
| Murba1 | MBP3303338.1 | 2 | Muribaculaceae bacterium |
| Bbac1 | MBR5851405.1 | 3 | Bacteroidaceae bacterium |
| Para1 | WP_147381213.1 | 4 | Parabacteroides sp. AM08-6 |
| FutA | WP_064780098.1 | 5 | Helicobacter pylori ATCC |
| | | | 26695 |

¹The GenBank IDs reflect the full-length enzymes, in the present invention truncated or mutated versions may have been used, these are represented by the sequences indicated by the SEQ ID NOs.

Example 1 described herein discloses the identification of the heterologous α-1,3(4)-fucosyltransferases Bgall1, Murba1 Bbac1 and Para1 (SEQ ID NO: 1, 2, 3 and 4 respectively), which are each capable of producing mixtures of HMOs comprising LNDFH-II when introduced into an LNT producing cell. Specifically, the three novel enzymes Bgall1, Murba1 and Bbac1 can to a varying extend transfer a fucosyl unit onto the Glc moiety of LNT in an α-1,3 linkage to form LNFP-V and further transfer a fucosyl unit onto the GlcNAc moiety of LNFP-V in an α-1,4 linkage to form LNDFH-II. Surprisingly example 1 also shows the FutA is capable of transfer a fucosyl unit onto the GlcNAc moiety of LNFP-V in an α-1,4 linkage to form LNDFH-II, which to our knowledge has not been shown previously.

The heterologous α-1,3(4)-fucosyltransferase Para1 appears to have a bit different specificity than the four other the heterologous α-1,3(4)-fucosyltransferase in table 1 in that it is capable of forming both LNFP-II and LNFP-V from LNT in similar amounts indicating it has similar specificity towards the Glc and GlcNAc moiety in LNT as well as to the Glc moiety of LNFP-II and GlcNAc moiety of LNFP-V, and can produce LNDFH-II from both molecules, (see FIG. 1).

In summary Example 1 show that the enzymes Bgall1, Murba1, Bbac1, and FutA do not produce any LNFP-II, or at least not any detectable amount of LNFP-II, as a final product. In contrast the enzyme Para1 from Parabacteroides sp. AM08-6 was found to produce both LNFP-II along with LNFP-V and LNDFH-II as final products.

A fucosyl transferase described herein is capable of transferring a fucosyl moiety from a fucosyl donor to an acceptor oligosaccharide into an α-1,3 linkage or an α-1,4 linkage. Such an enzyme is also known as an α-1,3(4)-fucosyltransferase. In preferred embodiments, a fucosyl transferase described herein is a fucosyltransferase with α-1,3-fucosyltransferase and α-1,4-fucosyltransferase activity, which is capable of fucosylating an oligosaccharide at a GlcNAc moiety and a Glc moiety.

In embodiments, the fucosyltransferase with α-1,3(4)-fucosyltransferase activity is selected from the group consisting of Bgall1, comprising or consisting of an amino acid sequence according to SEQ ID NO: 1 or a functional homologue thereof with an amino acid sequence that is at least 80% identical to SEQ ID NO: 1, Murba1 comprising or consisting of an amino acid sequence according to SEQ ID NO: 2 or a functional homologue thereof with an amino acid sequence that is at least 80% identical to SEQ ID NO: 2 and Bbac1 comprising or consisting of an amino acid sequence according to SEQ ID NO: 3, or a functional homologue thereof with an amino acid sequence that is at least 80% identical to SEQ ID NO: 3.

In embodiments, the fucosyltransferase with α-1,3(4)-fucosyltransferase activity is selected from the group consisting of Bgall1, comprising or consisting of an amino acid sequence according to SEQ ID NO: 1 or a functional homologue thereof with an amino acid sequence that is at least 80% identical to SEQ ID NO: 1 and Murba1 comprising or consisting of an amino acid sequence according to SEQ ID NO: 2 or a functional homologue thereof with an amino acid sequence that is at least 80% identical to SEQ ID NO: 2.

In one embodiment, the fucosyltransferase with α-1,3(4)-fucosyltransferase activity is Bgall1 from Bacteroides gallinaceum comprising or consisting of the amino acid sequence of SEQ ID NO: 1 or an amino acid sequence with at least 80%, such as at least 85%, such as at least 90%, such as at least 95%, or such as at least 99% sequence identity to SEQ ID NO: 1.

In another embodiment, the fucosyltransferase with α-1,3(4)-fucosyltransferase activity is Murba1 from Muribaculaceae bacterium comprising or consisting of the amino acid sequence of SEQ ID NO: 2 or an amino acid sequence with at least 80%, such as at least 85%, such as at least 90%, such as at least 95%, or such as at least 99% sequence identity to SEQ ID NO: 2.

In another embodiment, the fucosyltransferase with α-1,3(4)-fucosyltransferase activity is Bbac1 from Bacteroidaceae bacterium comprising or consisting of the amino acid sequence of SEQ ID NO: 3 or an amino acid sequence with at least 80%, such as at least 85%, such as at least 90%, such as at least 95%, or such as at least 99% sequence identity to SEQ ID NO: 3.

In another embodiment, the fucosyltransferase with α-1,3(4)-fucosyltransferase activity is Para1 from Parabacteroides sp. AM08-6 comprising or consisting of the amino acid sequence of SEQ ID NO: 4 or an amino acid sequence with at least 80%, such as at least 85%, such as at least 90%, such as at least 95%, or such as at least 99% sequence identity to SEQ ID NO: 4.

In another embodiment, the fucosyltransferase with α-1,3(4)-fucosyltransferase activity is FutA from Helicobacter pylori ATCC 26695 comprising or consisting of the amino acid sequence of SEQ ID NO: 5 or an amino acid sequence with at least 80%, such as at least 85%, such as at least 90%, such as at least 95%, or such as at least 99% sequence identity to SEQ ID NO: 5.

In one embodiment, the enzyme Bgall1 is introduced into a genetically engineered cell which further comprises a β-1,4-galactosyltransferase and preferably also a β-1,3-N-acetyl-glucosaminyl-transferase.

In one embodiment, the enzyme Murba1 is introduced into a genetically engineered cell which further comprises a β-1,3-galactosyltransferase and preferably also a β-1,3-N-acetyl-glucosaminyl-transferase.

In one embodiment, the enzyme Bbac1 is introduced into a genetically engineered cell which further comprises a β-1,3-galactosyltransferase and preferably also a β-1,3-N-acetyl-glucosaminyl-transferase.

In one embodiment, the enzyme Para1 is introduced into a genetically engineered cell which further comprises a β-1,3-galactosyltransferase and preferably also a β-1,3-N-acetyl-glucosaminyl-transferase.

In one embodiment, the enzyme FutA is introduced into a genetically engineered cell which further comprises a β-1,3-galactosyltransferase and preferably also a β-1,3-N-acetyl-glucosaminyl-transferase.

### β-1,3-N-Acetyl-Glucosaminyl-Transferase

A β-1,3-N-acetyl-glucosaminyl-transferase is any protein which comprises the ability of transferring the N-acetyl-glucosamine of UDP-N-acetyl-glucosamine to lactose or another acceptor molecule, in a β-1,3-linkage (see FIG. 1). Preferably the β-1,3-N-acetyl-glucosaminyl-transferase used herein does not originate in the species of the genetically engineered cell, i.e., the gene encoding the β-1,3-N-acetyl-glucosaminyl-transferase is of heterologous origin. In the context described herein, the acceptor molecule is either lactose or an oligosaccharide of at least four monosaccharide units, e.g., LNT, or more complex HMO structures.

Accordingly, in embodiments, the genetically engineered cell further comprises one or more recombinant nucleic acid sequence(s) encoding a β-1,3-N-acetyl-glucosaminyltransferase.

Non-limiting examples of β-1,3-N-acetyl-glucosaminyltransferases are given in table 2. β-1,3-N-acetyl-glucosaminyltransferase variants may also be useful, preferably such variants are at least 80%, such as at least 85%, such as at least 90%, such as at least 95% identical to the amino acid sequence of any one of the β-1,3-N-acetyl-glucosaminyltransferase in table 2.

TABLE 2
List of β-1,3-N-acetyl-glucosaminyltransferase

| Protein Name | GenBank ID | origin |
|---|---|---|
| lgtA | WP_033911473.1 | Neisseria meningitidis |
| | WP_002248149 | |
| | AAF42258.1 | |
| | ELK60643.1 | |
| lgtA | AAK70338.1 | Neisseria gonorrhoeae |
| | ACF31229.1 | |
| lgtA | AAK02595.1 | Pasteurella multocida |
| lgtA | EEZ72046.1 | Neisseria cinerea |
| HD0466 | WP_010944479.1 | Haemophilus ducreyi |
| PmnagT | WP_014390683.1 | Pasteurella multocida |

In embodiments, the genetically engineered cell comprises a recombinant nucleic acid sequence encoding α-1,3-N-acetyl-glucosaminyltransferase. In one embodiment, the recombinant nucleic acid sequence encoding a β-1,3-N-acetylglucosaminyltransferase comprises or consists of the amino acid sequence of SEQ ID NO: 12 (LgtA from N. meningitidis) or a functional homologue thereof with an amino acid sequence with at least 80%, such as at least 85%, such as at least 90%, such as at least 95%, or such as at least 99% sequence identity to SEQ ID NO: 12.

For the production of LNT from lactose as substrate, the LNT-II precursor is formed using a β-1,3-N-acetylglucosaminyltransferase. In one embodiment the genetically engineered cell comprises a β-1,3-N-acetylglucosaminyltransferase gene, or a functional homologue or fragment thereof, to produce the intermediate LNT-II from lactose.

Some of the examples below use the heterologous β-1,3-N-acetyl-glucosaminyl-transferase named LgtA from Neisseria meningitidis or a variant thereof.

### β-1,3-Galactosyltransferase

A β-1,3-galactosyltransferase is any protein that comprises the ability of transferring the galactose of UDP-Galactose to a N-acetyl-glucosaminyl moiety to an acceptor molecule in a β-1,3-linkage (see FIG. 1). Preferably, a β-1,3-galactosyltransferase used herein does not originate in the species of the genetically engineered cell i.e., the gene encoding the β-1,3-galactosyltransferase is of heterologous origin. As described herein the acceptor molecule, is an acceptor saccharide, e.g., LNT-II, or more complex HMO structures.

The examples below use the heterologous β-1,3-galactosyltransferase named GalTK or a variant thereof, to produce LNT and in in combination with α-1,3(4)-fucosyltransferase described herein it can produce e.g., LNFP-II, LNFP-V and/or LNDFH-II.

β-1,3-galactosyltransferases can be obtained from any one of a number of sources, e.g., the galTK gene from H. pylori as described, (homologous to GenBank protein Accession BD182026.1) or the WbgO gene from E. coli 055:H7 (GenBank Accession WP_000582563.1) or the jhp0563 gene from H. pylori (GenBank Accession AEZ55696.1).

In one embodiment, the recombinant nucleic acid sequence encoding a β-1,3-galactosyltransferases comprises or consists of the amino acid sequence of SEQ ID NO: 13 (galTK from H. pylori) or a functional homologue thereof with an amino acid sequence with at least 80%, such as at least 85%, such as at least 90%, such as at least 95%, or such as at least 99% identity to SEQ ID NO: 13.

To produce LNT form an LNT-II precursor, a β-1,3-galactosyltransferase is needed. In one embodiment, the genetically modified cell comprises a β-1,3-galactosyltransferase gene, or a functional homologue or fragment thereof.

Below are examples of genetically modified strains described herein with specific combinations of glycosyl transferases that will lead to production of LNDFH-II using lactose as initial substrate.

In one example, LgtA from Neisseria meningitidis is used in combination with galTK from Helicobacter pylori and Bgall1 from Bacteroides gallinaceum to produce LNDFH-II starting from lactose as initial substrate.

In one example, LgtA from Neisseria meningitidis is used in combination with galTK from Helicobacter pylori and Murba1 from Muribaculaceae bacterium to produce LNDFH-II starting from lactose as initial substrate.

In one example, LgtA from Neisseria meningitidis is used in combination with galTK from Helicobacter pylori and Bbac1 from Bacteroidaceae bacterium to produce LNDFH-II starting from lactose as initial substrate.

In one example, LgtA from Neisseria meningitidis is used in combination with galTK from Helicobacter pylori and Para1 from Parabacteroides sp. AM08-6 to produce LNDFH-II starting from lactose as initial substrate.

In one example, LgtA from Neisseria meningitidis is used in combination with galTK from Helicobacter pylori and FutA from Helicobacter pylori ATCC 26695 to produce LNDFH-II starting from lactose as acceptor saccharide.

In one example, galTK from Helicobacter pylori is used in combination with Bgall1 from Bacteroides gallinaceum to produce LNDFH-II starting from LNT-II as initial substrate.

In one example, galTK from Helicobacter pylori is used in combination with Murba1 from Muribaculaceae bacterium to produce LNDFH-II starting from LNT-II as initial substrate.

In one example, galTK from Helicobacter pylori is used in combination Bbac1 from Bacteroidaceae bacterium to produce LNDFH-II starting from LNT-II as initial substrate.

In one example, galTK from Helicobacter pylori and Para1 from Parabacteroides sp. AM08-6 to produce LNDFH-II starting from LNT-II as initial substrate.

In one example, galTK from Helicobacter pylori is used in combination with FutA from Helicobacter pylori ATCC 26695 to produce LNDFH-II starting from LNT-II as initial substrate.

### Glycosyl-Donor—Nucleotide-Activated Sugar Pathways

When carrying out the method of this invention, preferably a glycosyltransferase mediated glycosylation reaction takes place in which an activated sugar nucleotide serves as glycosyl-donor. An activated sugar nucleotide generally has a phosphorylated glycosyl residue attached to a nucleoside. A specific glycosyl transferase enzyme accepts only a specific sugar nucleotide. Thus, preferably the following activated sugar nucleotides are involved in the glycosyl transfer: glucose-UDP-GlcNAc, UDP-galactose, UDP-glucose, UDP-N-acetylglucosamine, UDP-N-acetylgalactosamine (GlcNAc) and CMP-N-acetylneuraminic acid.

The genetically engineered cell described herein can comprise one or more pathways to produce a nucleotide-activated sugar selected from the group consisting of glucose-UDP-GlcNAc, GDP-fucose, UDP-galactose, UDP-glucose, UDP-N-acetylglucosamine, UDP-N-acetylgalactosamine and CMP-N-acetylneuraminic acid.

In one embodiment of the current invention, the genetically engineered cell is capable of producing one or more activated sugar nucleotides mentioned above by a de novo pathway. In this regard, an activated sugar nucleotide is made by the cell under the action of enzymes involved in the de novo biosynthetic pathway of that respective sugar nucleotide in a stepwise reaction sequence starting from a simple carbon source like glycerol, sucrose, fructose or glucose (for a review for monosaccharide metabolism see e.g. H. H. Freeze and A. D. Elbein: Chapter 4: Glycosylation precursors, in: Essentials of Glycobiology, 2nd edition (Eds. A. Varki et al.), Cold Spring Harbour Laboratory Press (2009)).

The enzymes involved in the de novo biosynthetic pathway of an activated sugar nucleotide can be naturally present in the cell or introduced into the cell by means of gene technology or recombinant DNA techniques, all of them are parts of the general knowledge of the skilled person.

In another embodiment, the genetically engineered cell can utilize salvaged monosaccharides for sugar nucleotide. In the salvage pathway, monosaccharides derived from degraded oligosaccharides are phosphorylated by kinases, and converted to nucleotide sugars by pyrophosphorylases. The enzymes involved in the procedure can be heterologous ones, or native ones of the host cell.

### Colanic Acid Gene Cluster

For the production of fucosylated HMOs, the de novo GDP-fucose pathway is important to ensure presence of sufficient GDP-fucose. The colanic acid gene cluster of Escherichia coli encodes selected enzymes involved in the de novo synthesis of GDP-fucose (gmd, wcaG, wcaH, wcaI, manB, manC), whereas one or several of the genes downstream of GDP-L-fucose such as wcaJ, which are responsible for the production of the extracellular polysaccharide colanic acid, a major oligosaccharide of the bacterial cell wall, can be deleted to prevent conversion of GDP-fucose to colanic acid.

To secure sufficient amounts of GDP-fucose the promoter of the native colanic acid gene cluster may be exchanged with a stronger promoter, generating a recombinant colanic acid gene cluster, to drive additional production of GDP-fucose. Furthermore, an extra copy of the colanic acid gene cluster or selected genes thereof can be introduced in the genetically engineered cells as described in the examples.

In embodiments, the colanic acid gene cluster may be expressed from its native genomic locus. The expression may be actively modulated. The expression can be modulated by swapping the native promoter with a promoter of interest, and/or increasing the copy number of the colanic acid genes coding said protein(s) by expressing the gene cluster from another genomic locus than the native, or episomally expressing the colanic acid gene cluster or specific genes thereof.

In relation to the present disclosure, the term “native genomic locus”, in relation to the colanic acid gene cluster, relates to the original and natural position of the gene cluster in the genome of the genetically engineered cell.

The de novo GDP-fucose pathway genes responsible for the formation of GDP-fucose comprises or consists of the following genes:
i) manA which encodes the protein mannose-6 phosphate isomerase (EC 5.3.1.8, UniProt accession nr. P00946), which facilitates the interconversion of fructose 6-phosphate (F6P) and mannose-6-phosphate;
ii) manB which encodes the protein phosphomannomutase (EC 5.4.2.8, UniProt accession nr P24175), which is involved in the biosynthesis of GDP-mannose by catalyzing conversion mannose-6-phosphate into mannose-1-phosphate;
iii) manC which encodes the protein mannose-1-phosphate guanylyltransferase guanylyltransferase (EC: 2.7.7.13, UniProt accession nr P24174), which is involved in the biosynthesis of GDP-mannose through synthesis of GDP-mannose from GTP and α-D-mannose-1-phosphate;
iv) gmd which encodes the protein GDP-mannose-4,6-dehydratase (UniProt accession nr P0AC88), which catalyzes the conversion of GDP-mannose to GDP-4-dehydro-6-deoxy-D-mannose;
v) wcaG (fcl) which encodes the protein GDP-L-fucose synthase (EC 1.1.1.271, UniProt accession nr P32055) which catalyses the two-step NADP-dependent conversion of GDP-4-dehydro-6-deoxy-D-mannose to GDP-fucose.

Accordingly, it is preferred that the genetically engineered cell, when producing one or more fucosylated heterologous products, overexpresses either the entire colonic acid gene cluster (e.g. as identified in SEQ ID NO: 11 or a functional variant thereof) and/or one or more genes of the de novo GDP-fucose pathway selected from the group consisting of manA, manB, manC, gmd and wcaG.

### Lactose Permease

Lactose permease is a membrane protein which is a member of the major facilitator superfamily and can be classified as a symporter, which uses the proton gradient towards the cell to transport β-galactosides such as lactose in the same direction into the cell. In oligosaccharide-production, especially in the production of human milk oligosaccharides (HMOs), lactose is often the initial substrate being decorated to produce any HMO of interest in a bioconversion that happens in the cell interior. Thus, in the production of HMOs, there is a desire to be able to import lactose into the cell, e.g., by expression and/or overexpression of a lactose permease such as lacY of E. coli.

In embodiments, the lactose permease is as shown in SEQ ID NO: 14, or a functional homologue thereof having an amino acid sequence which is at least 80% identical, such as at least 85%, 90% or 95% identical to SEQ ID NO: 14.

In embodiments, the expression of the lactose permease is regulated by a promoter described herein.

### β-Galactosidase

A host cell suitable for HMO production, e.g., E. coli, may comprise an endogenous β-galactosidase gene or an exogenous β-galactosidase gene, e.g., E. coli comprises an endogenous lacZ gene (e.g., GenBank Accession Number V00296 (GI:41901)). For the purposes of the invention, when producing an HMO, it is preferred that the genetically engineered cell does not express a functional β-galactosidase to avoid the degradation of lactose if lactose is used as the initial substrate for producing the complex fucosylated HMO. In embodiments the lacZ gene may be inactivated by a complete or partial deletion of the corresponding nucleic acid sequence from the bacterial genome, or the gene sequence is mutated in the way that it is not transcribed, or, if transcribed, the transcript is not translated or if translated to a protein (i.e., β-galactosidase), the protein does not have the corresponding enzymatic activity. In this way the HMO-producing bacterium accumulates an increased intracellular lactose pool which is beneficial for the production of HMOs.

### Importer Proteins

Most commonly HMO producing cells are genetically engineered to use lactose as the initial substrate since this is easily taken up by lactose permease as described above. However, it may be desired to use an initial substrate that will require the presence of fewer glycosyltransferases in the cell, since this will reduce the strain on the cell in terms of producing multiple enzymes and in addition it can reduce the by-product profile, e.g. if lactose is not used as initial substrate a cell comprising a fucosyltransferase will not produce 3FL as by-product allowing the fucose to be used to produce e.g. more LNFP-V and LNDFH-II. WO2022/242860 suggests how it may be possible to identify LNT-II importers. WO2023/099680 also suggests a number of potential LNT and LNT-II importers. Examples of suitable LNT-II importers are e.g.,
Lactose permease (LacY) mutants, such as LacY mutant Y236H or LacY mutant A177V+S306T, wherein the mutations are equivalent with the corresponding position in the sequence of SEQ ID NO: 14,
ABC transporter protein complexes, such as ABC transporter from B. pseudocatenulatum JCM 1200 BBPC_1775, 1776, 1777, (NCBI accession Nrs BAR04453.1, BAR04454.1 and BAR04455.1, respectively) or ABC transporter from B. breve UCC2003 BBR_0527/IntP1, BBR_0528/IntP2, BBR_0530/IntS and BBR_0531 (NCBI accession Nrs ABE95224.1, ABE95225.1, ABE95226.1 and ABE95228.1), and/or
MFS transporters, such as but not limited to Blon_0962 (NCBI accession Nr ACJ52061.1).

Accordingly, in embodiments, a nucleic acid or a cluster of nucleic acids encoding one of these transporters may be introduced into a genetically modified cell as described herein. The expression of such transporters enables the production of complex fucosylated oligosaccharide with LNT-II as the initial substrate.

### Exporter Proteins

The oligosaccharide product, such as the HMO produced by the cell, can be accumulated both in the intra- and the extracellular matrix. The product can be transported to the supernatant in a passive way, i.e., it diffuses outside across the cell membrane. The more complex HMO products may remain in the cell, which is likely to eventually impair cellular growth, thereby affecting the possible total yield of the product from a single fermentation. The HMO transport can be facilitated by major facilitator superfamily transporter proteins that promote the effluence of sugar derivatives from the cell to the supernatant. The exporter can be present exogenously or endogenously and is overexpressed under the conditions of the fermentation to enhance the export of the oligosaccharide derivative (HMO) produced. The specificity towards the oligosaccharide product to be secreted can be altered by mutation by means of known recombinant DNA techniques.

Thus, the genetically engineered cell described herein can further comprise a nucleic acid sequence encoding an exporter protein capable of exporting the fucosylated human milk oligosaccharide product or products, such an exporter protein can for example be a member of the major facilitator superfamily transport proteins.

In the resent years, several new and efficient major facilitator superfamily transporter proteins have been identified as exporters of HMOs, each having specificity for different recombinantly produced HMOs and development of recombinant cells expressing said proteins are advantageous for high scale industrial HMO manufacturing.

### The Genetically Engineered Cell

In the present context, the terms “a genetically engineered cell” and “a genetically modified cell” are used interchangeably. As used herein “a genetically engineered cell” is a host cell whose genetic material has been altered by human intervention using a genetic engineering technique, such a technique is e.g., but not limited to transformation or transfection e.g., with a heterologous and/or recombinant polynucleotide sequence, Crisper/Cas editing and/or random mutagenesis. In one embodiment the genetically engineered cell has been transformed or transfected with a recombinant nucleic acid sequence.

The genetic modifications can e.g., be selected from inclusion of glycosyltransferases, and/or metabolic pathway engineering, deletion of repressors or undesired enzymes and inclusion of transporters as described in the above sections, which the skilled person will know how to combine into a genetically engineered cell capable of producing one or more fucosylated HMO's.

In one embodiment the genetically engineered cell capable of producing LNDFH-II, comprises a recombinant nucleic acid sequence encoding a fucosyltransferase with α-1,3(4)-fucosyltransferase activity, wherein said enzyme is selected from the group consisting of a) Bgall1, comprising or consisting of an amino acid sequence according to SEQ ID NO: 1 or a functional homologue thereof with an amino acid sequence that is at least 80% identical to SEQ ID NO: 1, b) Murba1 comprising or consisting of an amino acid sequence according to SEQ ID NO: 2 or a functional homologue thereof with an amino acid sequence that is at least 80% identical to SEQ ID NO: 2, c) Bbac1 comprising or consisting of an amino acid sequence according to SEQ ID NO: 3, or a functional homologue thereof with an amino acid sequence that is at least 80% identical to SEQ ID NO: 3 d) Para1 comprising or consisting of an amino acid sequence according to SEQ ID NO: 4, or a functional homologue thereof with an amino acid sequence that is at least 80% identical to SEQ ID NO: 4 and e) FutA comprising or consisting of an amino acid sequence according to SEQ ID NO: 5, or a functional homologue thereof with an amino acid sequence that is at least 80% identical to SEQ ID NO: 5.

Preferably, the fucosyltransferases have a dual α-1,3-fucosyltransferase and α-1,4-fucosyltransferase activity, allowing fucosylation of an oligosaccharide at position 4 of a GlcNAc moiety and at position 3 of a Glc moiety, while at the same time showing limited or no fucosylation at position 2 of the Gal moiety. Preferably, the Glc moiety is at the reducing end of the oligosaccharide, more preferably the oligosaccharide is LNT.

In one embodiment the genetically engineered cell capable of producing one or more fucosylated HMO, comprises a recombinant nucleic acid sequence encoding a fucosyltransferase with α-1,3(4)-fucosyltransferase activity, wherein said enzyme is selected from the group consisting of a) Bgall1, comprising or consisting of an amino acid sequence according to SEQ ID NO: 1 or a functional homologue thereof with an amino acid sequence that is at least 80% identical to SEQ ID NO: 1, b) Murba1 comprising or consisting of an amino acid sequence according to SEQ ID NO: 2 or a functional homologue thereof with an amino acid sequence that is at least 80% identical to SEQ ID NO: 2, c) Bbac1 comprising or consisting of an amino acid sequence according to SEQ ID NO: 3, or a functional homologue thereof with an amino acid sequence that is at least 80% identical to SEQ ID NO: 3.

In another embodiment the genetically engineered cell capable of producing one or more fucosylated HMOs of which at least one is LNDFH-II, comprises a recombinant nucleic acid sequence encoding a fucosyltransferase with α-1,3(4)-fucosyltransferase activity, wherein said enzyme is Para1 comprising or consisting of an amino acid sequence according to SEQ ID NO: 4, or a functional homologue thereof with an amino acid sequence that is at least 80% identical to SEQ ID NO: 4.

In another embodiment the genetically engineered cell capable of producing one or more fucosylated HMOs of which at least one is LNDFH-II, comprises a recombinant nucleic acid sequence encoding a fucosyltransferase with α-1,3(4)-fucosyltransferase activity, wherein said enzyme is FutA comprising or consisting of an amino acid sequence according to SEQ ID NO: 5, or a functional homologue thereof with an amino acid sequence that is at least 80% identical to SEQ ID NO: 5. In some embodiments, it is advantageous for the genetically modified cell to have a high expression of the FutA, this can for example be achieved by having at least two copies of the recombinant nucleic acid sequence encoding FutA (SEQ ID NO: 5) or a functional variant thereof on the genome, or by introducing a high copy nr. plasmid encoding FutA (SEQ ID NO: 5) or a functional variant thereof and/or by controlling the expression from strong or very strong promoters.

In preferred embodiments the genetically engineered cell capable of producing LNDFH-II, comprises a recombinant nucleic acid sequence encoding a fucosyltransferase with α-1,3(4)-fucosyltransferase activity, wherein said enzyme is selected from the group consisting of a) Bgall1, comprising or consisting of an amino acid sequence according to SEQ ID NO: 1 or a functional homologue thereof with an amino acid sequence that is at least 80% identical to SEQ ID NO: 1, b) Murba1 comprising or consisting of an amino acid sequence according to SEQ ID NO: 2 or a functional homologue thereof with an amino acid sequence that is at least 80% identical to SEQ ID NO: 2 and c) Bbac1 comprising or consisting of an amino acid sequence according to SEQ ID NO: 3, or a functional homologue thereof with an amino acid sequence that is at least 80% identical to SEQ ID NO: 3.

In a presently preferred embodiments, the genetically engineered cell capable of producing LNDFH-II, comprises a recombinant nucleic acid sequence encoding a fucosyltransferase with α-1,3(4)-fucosyltransferase activity, wherein said enzyme is Bgall1, comprising or consisting of an amino acid sequence according to SEQ ID NO: 1 or a functional homologue thereof with an amino acid sequence that is at least 80% identical to SEQ ID NO: 1. [Bgall1 amino acid].

In one aspect of the invention, the genetically engineered cell comprises a recombinant nucleic acid sequence encoding a fucosyltransferase with α-1,3(4)-fucosyltransferase activity, which is capable of producing at least 1 molar % LNDFH-II of the total molar HMO content produced by the cell. Preferably, the at least 5 molar % of the molar content of the total HMOs produced by said cell is LNDFH-II. In embodiments, at least at least 5 molar %, such as at least 5 molar %, 6 molar %, 7 molar %, 8 molar %, 9 molar %, 10 molar %, 11 molar %, 12 molar %, 13 molar %, 14 molar %, 15 molar %, 20 molar %, 25 molar %, 30 molar %, or such as at least 35 molar % of the molar content of the total HMOs produced by said cell is LNDFH-II. In additional embodiments, the cell further produces one or more HMOs selected from the group consisting of 3FL, LNT and LNFP-V.

In some embodiments, the genetically engineered cell described herein expresses Bgall1 comprising or consisting of the amino acid sequence of SEQ ID NO: 1 or an amino acid sequence with at least 80%, such as at least 85%, such as at least 90%, such as at least 95%, or such as at least 99% sequence identity to SEQ ID NO: 1, and the molar % content of LNDFH-II produced by the genetically engineered cell is above 10 molar %, such as above 15 molar %, such as above 17 molar %, such as above 19 molar %, such as above 20 molar %, or such as above 23 molar % of the total HMO produced.

In some embodiments, the genetically engineered cell described herein expresses Murba1 comprising or consisting of the amino acid sequence of SEQ ID NO: 2 or an amino acid sequence with at least 80%, such as at least 85%, such as at least 90%, such as at least 95%, or such as at least 99% sequence identity to SEQ ID NO: 2, and the molar % content of LNDFH-II produced by the genetically engineered cell is above 1 molar %, such as above 3 molar %, such as above 4 molar %, such as above 5 molar %, or such as above 6 molar % of the total HMO produced.

In some embodiments, the genetically engineered cell described herein expresses Bbac1 comprising or consisting of the amino acid sequence of SEQ ID NO: 3 or an amino acid sequence with at least 80%, such as at least 85%, such as at least 90%, such as at least 95%, or such as at least 99% sequence identity to SEQ ID NO: 3, and the molar % content of LNDFH-II produced by the genetically engineered cell is above 1 molar %, such as above 3 molar %, such as above 4 molar %, such as above 5 molar %, or such as above 6 molar % of the total HMO produced.

In some embodiments, the genetically engineered cell described herein expresses Para1 comprising or consisting of the amino acid sequence of SEQ ID NO: 4 or an amino acid sequence with at least 80%, such as at least 85%, such as at least 90%, such as at least 95%, or such as at least 99% sequence identity to SEQ ID NO: 4, and the molar % content of LNDFH-II produced by the genetically engineered cell is above 1 molar %, such as above 3 molar %, such as above 4 molar %, such as above 5 molar %, or such as above 6 molar % of the total HMO produced.

In some embodiments, the genetically engineered cell described herein expresses FutA comprising or consisting of the amino acid sequence of SEQ ID NO: 5 or an amino acid sequence with at least 80%, such as at least 85%, such as at least 90%, such as at least 95%, or such as at least 99% sequence identity to SEQ ID NO: 5, and the molar % content of LNDFH-II produced by the genetically engineered cell is above 4 molar %, such as above 10 molar %, such as above 15 molar %, such as above 20 molar %, such as above 25 molar %, or such as above 35 molar % of the total HMO produced.

More than 60% of the HMOs found in human milk are different species of fucosylated HMOs. Accordingly, in embodiments, at least 60%, such as at least 75%, 80%, 85%, 90%, 95% or at least 97% of the molar content of the total HMOs produced by said cell are fucosylated. Preferably, the fucosylated HMOs produced by the cell are selected from the group consisting of 3FL, LNFP-V and LNDFH-II. 1. In one embodiment the genetically engineered cell described herein produces a mixture of 3FL, LNFP-V and LNDFH-II wherein the mixture constitutes at least 90 molar % of the total HMO produced by the cell.

In some embodiments, the genetically engineered cell described herein expresses Bgall1 comprising or consisting of the amino acid sequence of SEQ ID NO: 1 or an amino acid sequence with at least 80%, such as at least 85%, such as at least 90%, such as at least 95%, or such as at least 99% sequence identity to SEQ ID NO: 1, and the molar % content of fucosylated HMOs produced by the genetically engineered cell is above 85 molar %, such as above 87 molar %, such as above 89 molar %, such as above 90 molar %, such as above 93 molar %, or such as above 96 molar % of the total HMO produced.

In some embodiments, the genetically engineered cell described herein expresses Murba1 comprising or consisting of the amino acid sequence of SEQ ID NO: 2 or an amino acid sequence with at least 80%, such as at least 85%, such as at least 90%, such as at least 95%, or such as at least 99% sequence identity to SEQ ID NO: 2, and the molar % content of fucosylated HMOs produced by the genetically engineered cell is above 75 molar %, such as above 77 molar %, such as above 80 molar %, such as above 85 molar %, such as above 90 molar %, or such as above 95 molar % of the total HMO produced.

In some embodiments, the genetically engineered cell described herein expresses Bbac1 comprising or consisting of the amino acid sequence of SEQ ID NO: 3 or an amino acid sequence with at least 80%, such as at least 85%, such as at least 90%, such as at least 95%, or such as at least 99% sequence identity to SEQ ID NO: 3, and the molar % content of fucosylated HMOs produced by the genetically engineered cell is above 85 molar %, such as above 87 molar %, such as above 89 molar %, such as above 90 molar %, such as above 93 molar %, or such as above 96 molar % of the total HMO produced.

In some embodiments, the genetically engineered cell described herein expresses FutA comprising or consisting of the amino acid sequence of SEQ ID NO: 5 or an amino acid sequence with at least 80%, such as at least 85%, such as at least 90%, such as at least 95%, or such as at least 99% sequence identity to SEQ ID NO: 5, and the molar % content of fucosylated HMOs produced by the genetically engineered cell is above 85 molar %, such as above 87 molar %, such as above 89 molar %, such as above 90 molar %, such as above 93 molar %, or such as above 96 molar % of the total HMO produced.

In preferred embodiments, the fucosylated HMOs produced by a cell described herein are selected from the group consisting of 3FL, LNFP-V and LNDFH-II.

The genetically engineered cell described herein preferably expresses genes encoding key enzymes for the biosynthesis of fucosylated HMOs. In addition, it is advantageous if the genetically engineered cell expresses the genes needed to produce LNT, either from lactose or LNT-II as the initial substrate (see FIG. 1), and/or alternatively the cell expresses importers for LNT-II or LNT.

In embodiments the genetically engineered cell comprises one or more additional glycosyltransferases. The additional one or more glycosyltransferases are preferably selected from the group consisting of, galactosyltransferases, glucosaminyltransferases, fucosyltransferases and N-acetylglucosaminyl transferases.

In some embodiments the genetically engineered cell comprises one or more recombinant nucleic acid sequence(s) encoding a β-1,3-galactosyltransferase and optionally a β-1,3-N-acetylglucosaminyltransferase. In some embodiments the β-1,3-N-acetylglucosaminyltransferase is from Neisseria meningitidis, and the β-1,3-galactosyltransferase is from Helicobacter pylori.

In some embodiments, a genetically engineered cell described herein further expresses the de novo GDP-fucose pathway genes responsible for the formation of GDP-fucose manA, manB, manC, gmd and wcaG. It may be advantageous to overexpress one or more of these genes and/or to upregulate the colanic acid gene cluster (CA), including the genes gmd, wcaG, wcaH, wcaI, manC and manB from E. coli, through introduction of a nucleic acid construct encoding the CA as shown in SEQ ID NO: 11, allowing for formation of GDP-fucose, which enables the cell to produce a higher level of fucosylated oligosaccharides from one or more intermediate oligosaccharide substrates, such as lactose or LNT, LNFP-II and/or LNFP-V. Depending on the intended use of substrate, one or more additional glycosyltransferases and pathways for producing nucleotide-activated sugars, such as glucose-UDP-GlcNAc, CMP-N-acetylneuraminic acid, UDP-galactose, UDP-glucose, UDP-N-acetylglucosamine, UDP-N-acetylgalactosamine and/or CMP-N-acetylneuraminic acid can also be present in the genetically engineered cell.

It is further understood that the genetically engineered cell described herein may further comprise any of the modifications described above, e.g., additional glycosyltransferases, suitable importer proteins such as overexpression of lactose permease, LNT-II or LNT importers, β-galactosidase inactivation in particular if lactose is used as the initial substrate, as well suitable exporter proteins for the complex fucosylated HMOs produced by the cell.

### HMO Mixtures Produced by the Cell

The genetically engineered cell comprising an α-1,3(4)-fucosyltransferase described herein described herein with dual fucosyltransferase specificity will generally produce a mixture of HMOs as a result of the multistep process inside the cell towards the final HMO product, LNDFH-II (see FIG. 1). In the production of LNDFH-II from lactose as the initial substrate, it is expected that certain amounts of 3FL (fucosylated lactose), LNT-II, LNT, LNFP-V, and potentially also LNFP-II will be produced by the cell.

The molar % of individual HMO components supported by experimental data from the Examples shows exemplary HMO composition ranges, wherein the mixture of final HMOs products consists essentially of 3FL, LNT-II, LNT, LNFP-II, LNFP-V and LNDFH-II 3FL, or LNT-II, LNT, LNFP-V and LNDFH-II, where 3FL can be in very low amounts or not present at all.

In embodiments, the cell produces a final mixture comprising LNDFH-II, LNFP-V and LNT. In another embodiment, the cell produces a mixture of HMOs consisting essentially of a mixture selected from the group consisting of i) LNDFH-II, LNFP-V and LNT, ii) LNDFH-II, LNFP-V and 3FL, iii) LNDFH-II, LNFP-V, LNT and 3FL, iv) LNDFH-II, LNFP-V, LNFP-II, LNT and LNT-II, and v) LNDFH-II, LNFP-V, LNFP-II, LNT, LNT-II and 3FL. In another embodiment the cell produces a mixture consisting essentially of LNDFH-II, LNFP-V and 3FL. In another embodiment the cell produces a mixture consisting essentially of LNDFH-II, LNFP-V, LNT and 3FL. In another embodiment the cell produces a mixture consisting essentially of LNDFH-II, LNFP-V, LNFP-II, LNT and LNT-II.

In embodiments, the cell produces a mixture of HMOs consisting essentially of 1-40 molar % of LNDFH-II, 2-85 molar % LNFP-V, 0-10 molar % LNFP-II, 0-65 molar % 3FL, 0-550% LNT and 0-50 molar % LNT-II, in total adding up to 100% molar content.

In some embodiments, the genetically engineered cell described herein expresses Bgall1 comprising or consisting of the amino acid sequence of SEQ ID NO: 1 or an amino acid sequence with at least 80%, such as at least 85%, such as at least 90%, such as at least 95%, or such as at least 99% sequence identity to SEQ ID NO: 1 and the produced mixture consists essentially of 3FL, LNT, LNFP-V and LNDFH-II, wherein 15-40% of the total molar HMO content in the mixture is LNDFH-II, 20-60% of the total molar HMO content in the mixture is LNFP-V, 0-15% of the total molar HMO content in the mixture is LNT and 15-60% of the total molar HMO content in the mixture is 3FL, in total adding up to 100% molar content.

In some embodiments, the genetically engineered cell described herein expresses Murba1 comprising or consisting of the amino acid sequence of SEQ ID NO: 2 or an amino acid sequence with at least 80%, such as at least 85%, such as at least 90%, such as at least 95%, or such as at least 99% sequence identity to SEQ ID NO: 2 and the produced mixture consists essentially of 3FL, LNT, LNFP-V and LNDFH-II, wherein 1-15% of the total molar HMO content in the mixture is LNDFH-II, 35-70% of the total molar HMO content in the mixture is LNFP-V, 0-25% of the total molar HMO content in the mixture is LNT and 5-55% of the total molar HMO content in the mixture is 3FL, in total adding up to 100% molar content.

In some embodiments, the genetically engineered cell described herein expresses Bbac1 comprising or consisting of the amino acid sequence of SEQ ID NO: 3 or an amino acid sequence with at least 80%, such as at least 85%, such as at least 90%, such as at least 95%, or such as at least 99% sequence identity to SEQ ID NO: 3 and the produced mixture consists essentially of 3FL, LNT, LNFP-V and LNDFH-II, wherein 2-15% of the total molar HMO content in the mixture is LNDFH-II, 60-75% of the total molar HMO content in the mixture is LNFP-V, 0-10% of the total molar HMO content in the mixture is LNT and 15-30% of the total molar HMO content in the mixture is 3FL, in total adding up to 100% molar content.

In some embodiments, the genetically engineered cell described herein expresses Para1 comprising or consisting of the amino acid sequence of SEQ ID NO: 4 or an amino acid sequence with at least 80%, such as at least 85%, such as at least 90%, such as at least 95%, or such as at least 99% sequence identity to SEQ ID NO: 4 and the produced mixture consists essentially of 3FL, LNT, LNFP-II, LNFP-V and LNDFH-II, wherein 2-15% of the total molar HMO content in the mixture is LNDFH-II, 0-10% of the total molar HMO content in the mixture is LNFP-V, 0-10% of the total molar HMO content in the mixture is LNFP-II, 30-50% of the total molar HMO content in the mixture is LNT-II, 30-60% of the total molar HMO content in the mixture is LNT and 0-50% of the total molar HMO content in the mixture is 3FL, in total adding up to 100% molar content.

In some embodiments, the genetically engineered cell described herein expresses FutA comprising or consisting of the amino acid sequence of SEQ ID NO: 5 or an amino acid sequence with at least 80%, such as at least 85%, such as at least 90%, such as at least 95%, or such as at least 99% sequence identity to SEQ ID NO: 5 and the produced mixture consists essentially of 3FL, LNT, LNFP-V and LNDFH-II, wherein 2-40% of the total molar HMO content in the mixture is LNDFH-II, 35-85% of the total molar HMO content in the mixture is LNFP-V, 0-25% of the total molar HMO content in the mixture is LNT and 0-20% of the total molar HMO content in the mixture is 3FL, in total adding up to 100% molar content.

### Host Cells

In embodiments, the engineered cell is a microorganism. The genetically engineered cell is preferably a microbial cell, such as a prokaryotic cell or eukaryotic cell. Appropriate microbial cells that may function as a host cell include bacterial cells, archaebacterial cells, algae cells and fungal cells.

The genetically engineered cell may be e.g., a bacterial or yeast cell. In one preferred embodiment, the genetically engineered cell is a bacterial cell.

Regarding the bacterial host cells, there are, in principle, no limitations; they may be eubacteria (gram-positive or gram-negative) or archaebacteria, as long as they allow genetic manipulation for insertion of a gene of interest and can be cultivated on a manufacturing scale. Preferably, the host cell has the property to allow cultivation to high cell densities. Non-limiting examples of bacterial host cells that are suitable for recombinant industrial production of an HMO(s) according to the invention could be member of the Enterobacterales order, preferably of the genus Escherichia, more preferably of the species E. coli. Other examples of suitable host cell are Erwinia herbicola (Pantoea agglomerans), Citrobacter freundii, Campylobacter sp, Pantoea citrea, Pectobacterium carotovorum, or Xanthomonas campestris. Bacteria of the genus Bacillus may also be used, including Bacillus subtilis, Bacillus licheniformis, Bacillus coagulans, Bacillus thermophilus, Bacillus laterosporus, Bacillus megaterium, Bacillus mycoides, Bacillus pumilus, Bacillus lentus, Bacillus cereus, and Bacillus circulans. Similarly, bacteria of the genera Lactobacillus and Lactococcus may be engineered using the methods of this invention, including but not limited to Lactobacillus acidophilus, Lactobacillus salivarius, Lactobacillus plantarum, Lactobacillus helveticus, Lactobacillus delbrueckii, Lactobacillus rhamnosus, Lactobacillus bulgaricus, Lactobacillus crispatus, Lactobacillus gasseri, Lactobacillus casei, Lactobacillus reuteri, Lactobacillus jensenii, and Lactococcus lactis. Streptococcus thermophiles and Propionibacterium freudenreichii are also suitable bacterial species for the invention described herein. Also included as part of this invention as useful species are strains, engineered as described here, from the genera Enterococcus (e.g., Enterococcus faecium and Enterococcus thermophiles), Bifidobacterium (e.g., Bifidobacterium longum, Bifidobacterium infantis, and Bifidobacterium bifidum), Sporolactobacillus spp., Micromonospora spp., Micrococcus spp., Rhodococcus spp., and Pseudomonas (e.g., Pseudomonas fluorescens and Pseudomonas aeruginosa).

Non-limiting examples of fungal host cells that are suitable for recombinant industrial production of a heterologous product are e.g., yeast cells, such as Komagataella, Kluyveromyces, Yarrowia, Pichia, Saccaromyces, Schizosaccharomyces or Hansenula or from a filamentous fungus of the genera Aspergillus, Fusarium or Trichoderma.

In one or more exemplary embodiments, the genetically engineered cell is selected from the group consisting of Escherichia sp., Bacillus sp., Lactobacillus sp., Corynebacterium sp. and Campylobacter sp.

In one or more exemplary embodiments, the genetically engineered cell is selected from the group consisting of Escherichia coli, Bacillus subtilis, Lactobacillus lactis, Corynebacterium glutamicum, Yarrowia lipolytica, Pichia pastoris, and Saccharomyces cerevisiae.

In one or more exemplary embodiments, the genetically engineered cell is B. subtilis.

In one or more exemplary embodiments, the genetically engineered cell is S. Cerevisiae or P pastoris.

In one or more exemplary embodiments, the genetically engineered cell is Escherichia coli.

In one or more exemplary embodiments, the invention relates to a genetically engineered cell, wherein the cell is derived from the E. coli K-12 strain or DE3.

### a Recombinant Nucleic Acid Sequence

The present invention relates to a genetically engineered cell comprising a recombinant nucleic acid sequence encoding a fucosyltransferase with α-1,3(4)-fucosyltransferase activity, such as an enzyme selected from the group consisting of Bgall1, Bbac1, Murba1, Para1 and FutA, and wherein said cell produces Human Milk Oligosaccharides (HMO). In particular, at least one fucosylated HMO, and preferably with a molar % content of LNDFH-II above, or at least of 5% of the total HMO produced.

In the present context, the term “recombinant nucleic acid sequence”, “recombinant gene/nucleic acid/nucleotide sequence/DNA encoding” or “coding nucleic acid sequence” is used interchangeably and intended to mean an artificial nucleic acid sequence (i.e. produced in vitro using standard laboratory methods for making nucleic acid sequences) that comprises a set of consecutive, non-overlapping triplets (codons) which is transcribed into mRNA and translated into a protein when under the control of the appropriate control sequences, i.e., a promoter sequence.

The boundaries of the coding sequence are generally determined by a ribosome binding site located just upstream of the open reading frame at the 5′end of the mRNA, a transcriptional start codon (AUG, GUG or UUG), and a translational stop codon (UAA, UGA or UAG). A coding sequence can include, but is not limited to, genomic DNA, cDNA, synthetic, and recombinant nucleic acid sequences.

The term “nucleic acid” includes RNA, DNA and cDNA molecules. It is understood that, as a result of the degeneracy of the genetic code, a multitude of nucleic acid sequences encoding a given protein may be produced.

The recombinant nucleic acid sequence may be a coding DNA sequence e.g., a gene, or non-coding DNA sequence e.g., a regulatory DNA, such as a promoter sequence or other non-coding regulatory sequences.

The recombinant nucleic acid sequence may in addition be heterologous. As used herein “heterologous” refers to a polypeptide, amino acid sequence, nucleic acid sequence or nucleotide sequence that is foreign to a cell or organism, i.e., to a polypeptide, amino acid sequence, nucleic acid molecule or nucleotide sequence that does not naturally occurs in said cell or organism.

The invention also relates to a nucleic acid construct comprising a coding nucleic sequence, i.e. recombinant DNA sequence of a gene of interest, e.g., a fucosyltransferase gene, and a non-coding regulatory DNA sequence, e.g., a promoter DNA sequence, e.g., a recombinant promoter sequence derived from the promoter sequence of the lac operon or the glp operon, or a promoter sequence derived from another genomic promoter DNA sequence, or a synthetic promoter sequence, wherein the coding and promoter sequences are operably linked.

The term “operably linked” refers to a functional relationship between two or more nucleic acid (e.g., DNA) segments. It refers to the functional relationship of a transcriptional regulatory sequence to a transcribed sequence. E.g., a promoter sequence is operably linked to a coding sequence if it stimulates or modulates the transcription of the coding sequence in an appropriate host cell or other expression system.

Generally, promoter sequences that are operably linked to a transcribed sequence are physically contiguous to the transcribed sequence, i.e., they are cis-acting.

In one exemplified embodiment, the nucleic acid construct of the invention may be a part of the vector DNA, in another embodiment, the construct it is an expression cassette/cartridge that is integrated in the genome of a host cell.

Accordingly, the term “nucleic acid construct” means an artificially constructed segment of nucleic acids, in particular a DNA segment, which is intended to be inserted into a target cell, e.g., a bacterial cell, to modify expression of a gene of the genome or expression of a gene/coding DNA sequence which may be included in the construct. Thus, in embodiments, the present invention relates to a nucleic acid construct comprising a recombinant nucleic acid sequence encoding a fucosyltransferase, wherein said recombinant nucleic acid sequence is selected from the group consisting of nucleic acid sequences encoding Bgall1, Murba1, Bbac1, Para1 and FutA, such as a nucleic acid sequence according to SEQ ID NO: 6, 7, 8, 9 or 10, or functional variants thereof.

The genetically engineered cell described herein may also comprise multiple copies of the recombinant nucleic acid sequence encoding a fucosyltransferase. Enhancing the copy number of the fucosyltransferase was shown in Example 1 to change the ratio of the produced HMOs. Specifically, it was shown that increasing the copy number of Bgall1 by introduction of firstly two genomic copies and subsequently a high copy-number plasmid (pUC57-Bgall1-PglpF-amp), in the first instance resulted in an increase in between 1.5- and 1.8-fold for both 3FL and LNDFH-II and a 1.5-fold decrease in LNFP-V produced. Overexpression from a plasmid resulted in a slight increase in production of 3FL, combined with a decreased in both LNFP-V and LNDFH-II produced. Furthermore, increasing the copy number of Murba1 to two genomic copies and further to also include a high copy plasmid (pUC57-Murba1-PglpF-amp), increased the relative amount of LNDFH-II and 3FL produced, combined with a decrease in the amount of LNT and LNFP-V produced. The increase in copy nr of Para1 from one copy to two copies increased the relative amount of LNDFH-II produced by 5-fold, accompanied by an increase in the relative amount of LNT-II and a decrease in LNT. The increase in copy nr of FutA unexpectedly increased the relative amount of LNDFH-II 2.5-fold when adding one additional copy and almost 8-fold when expressed from a plasmid which was accompanied by an increase in the relative amount of 3FL production and a decrease in the relative amount of LNT and LNFP-V produced.

Accordingly, the copy number variation may be used in the production to tailor specific HMOs mixtures, in this case a mixture comprising 3FL, LNT-II, LNT, LNFP-V and LNDFH-II in different ratios, depending on the need for the specific product.

Accordingly, in embodiments, the genetically engineered cell described herein comprises one, two, three or more genomic copies of the recombinant nucleic acid sequence encoding the glycosyltransferase selected from the group consisting of Bgall1, Murba1, Bbac1 Para1 and FutA comprising or consisting of an amino acid sequence according to SEQ ID NO: 1, 2, 3, 4 or 5, or a functional homologue thereof with an amino acid sequence that is at least 80% identical to SEQ ID NO: 1, 2, 3, 4 or 5. In further embodiments, the recombinant nucleic acid sequence encoding the glycosyltransferase selected from the group consisting of Bgall1, Murba1, Bbac1, Para1 and FutA comprising or consisting of an amino acid sequence according to SEQ ID NO: 1, 2, 3, 4 or 5, or a functional homologue thereof with an amino acid sequence that is at least 80% identical to SEQ ID NO: 1, 2, 3, 4 or 5, is encoded on a plasmid. In additional embodiments, the plasmid is a high copy number plasmid, preferably, a pUC57 or pBB-B9 plasmid.

In additional embodiments the genetically engineered cell described herein comprises at least two genomic copies of a recombinant nucleic acid sequence encoding the fucosyltransferase FutA comprising or consisting of an amino acid sequence according to SEQ ID NO: 5, or a functional homologue thereof with an amino acid sequence that is at least 80% identical to SEQ ID NO: 5. In additional embodiments, said cell may further comprise at least one plasmid borne copy of the recombinant nucleic acid encoding the fucosyltransferase FutA.

In further embodiments, the genetically engineered cell described herein comprises one, two, three or more genomic copies and/or a plasmid borne copy of the recombinant nucleic acid sequence encoding the glycosyltransferase selected from the group consisting of Bgall1, Murba1 and Bbac1 comprising or consisting of an amino acid sequence according to SEQ ID NO: 1, 2, or 3, or a functional homologue thereof with an amino acid sequence that is at least 80% identical to SEQ ID NO: 1, 2, or 3.

One embodiment relates to a nucleic acid construct comprising a recombinant nucleic acid sequence encoding a fucosyltransferase, wherein said recombinant nucleic acid sequence is selected from the group consisting of a) Bgall1 comprising or consisting of the nucleic acid sequences of SEQ ID NO: 6 or an nucleic acid sequence with at least 80%, such as at least 85%, such as at least 90%, such as at least 95%, or such as at least 99% sequence identity to SEQ ID NO: 6; b) Murba1 comprising or consisting the nucleic acid sequences of SEQ ID NO: 7 or an nucleic acid sequence with at least 80%, such as at least 85%, such as at least 90%, such as at least 95%, or such as at least 99% sequence identity to SEQ ID NO: 7; c) Bbac1 comprising or consisting the nucleic acid sequence of SEQ ID NO: 8 or an nucleic acid sequence with at least 80%, such as at least 85%, such as at least 90%, such as at least 95%, or such as at least 99% sequence identity to SEQ ID NO: 8, and/or d) Para1 comprising or consisting the nucleic acid sequence of SEQ ID NO: 9 or an nucleic acid sequence with at least 80%, such as at least 85%, such as at least 90%, such as at least 95%, or such as at least 99% sequence identity to SEQ ID NO: 9 and e) FutA comprising or consisting the nucleic acid sequence of SEQ ID NO: 10 or an nucleic acid sequence with at least 80%, such as at least 85%, such as at least 90%, such as at least 95%, or such as at least 99% sequence identity to SEQ ID NO: 10.

Preferably, the fucosyltransferase encoding sequence is under the control of a promoter sequence selected from promotor sequences with a nucleic acid sequence as identified in Table 3.

TABLE 3
Selected promoter sequences

| | % Activity | | | Seq ID |
|---|---|---|---|---|
| Promoter name | relative to PglpF* | Strength | Reference | in appl. |
| PmglB_70UTR_SD8 | 291% | high | WO2020255054 | 15 |
| PmglB_70UTR_SD10 | 233-281% | high | WO2020255054 | 16 |
| PmglB_54UTR | 197% | high | WO2020255054 | 17 |
| Plac_70UTR | 182-220% | high | WO2019123324 | 18 |
| PmglB_70UTR_SD9 | 180-226% | high | WO2020255054 | 19 |
| PmglB_70UTR_SD4 | 153%-353% | high | WO2020255054 | 20 |
| PmglB_70UTR_SD5 | 146-152% | high | WO2020255054 | 21 |
| PglpF_SD4 | 140-161% | high | WO2019123324 | 22 |
| PmglB_70UTR_SD7 | 127-173% | high | WO2019123324 | 23 |
| PmglB_70UTR | 124-234% | high | WO2020255054 | 24 |
| PglpA_70UTR | 102-179% | high | WO2019123324 | 25 |
| PglpT_70UTR | 102-240% | high | WO2019123324 | 26 |
| PglpF | 100% | high | WO2019123324 | 27 |
| PglpF_SD10 | 88-96% | high | WO2019123324 | 28 |
| PglpF_SD5 | 82-91% | high | WO2019123324 | 29 |
| PglpF_SD8 | 81-82% | high | WO2019123324 | 30 |
| PmglB_16UTR | 78-171% | high | WO2019123324 | 31 |
| PglpF_SD9 | 73-93% | middle | WO2019123324 | 32 |
| PglpF_SD7 | 47-57% | middle | WO2019123324 | 33 |
| PglpF_SD6 | 46-47% | middle | WO2019123324 | 34 |
| PglpA_16UTR | 38-64% | middle | WO2019123324 | 35 |
| Plac_wt* | 15-28% | low | WO2019123324 | 36 |
| PglpF_SD3 | 9% | low | WO2019123324 | 37 |
| PglpF_SD1 | 5% | low | WO2019123324 | 38 |

*The promoter activity is assessed in the LacZ assay described below with the PglpF promoter run as positive reference in the same assay. To compare across assays the activity is calculated relative to the PglpF promoter, a range indicates results from multiple assays.

The promoter may be of heterologous origin, native to the genetically engineered cell or it may be a recombinant promoter, combining heterologous and/or native elements.

One way to increase the production of a product may be to regulate the production of the desired enzyme activity used to produce the product, such as the glycosyltransferases or enzymes involved in the biosynthetic pathway of the glycosyl donor.

Increasing the promoter strength driving the expression of the desired enzyme may be one way of doing this. The strength of a promoter can be assessed using a lacZ enzyme assay where β-galactosidase activity is assayed as described previously (see e.g., Miller J. H. Experiments in molecular genetics, Cold spring Harbor Laboratory Press, NY, 1972). Briefly the cells are diluted in Z-buffer and permeabilized with sodium dodecyl sulfate (0.1%) and chloroform. The LacZ assay is performed at 30° C. Samples are preheated, the assay initiated by addition of 200 μl ortho-nitro-phenyl-β-galactosidase (4 mg/ml) and stopped by addition of 500 μl of 1 M Na₂CO₃ when the sample had turned slightly yellow. The release of ortho-nitrophenol is subsequently determined as the change in optical density at 420 nm. The specific activities are reported in Miller Units (MU) [A420/(min*ml*A600)]. A regulatory element with an activity above 10,000 MU is considered strong and a regulatory element with an activity below 3,000 MU is considered weak, what is in between has intermediate strength. An example of a strong regulatory element is the PglpF promoter with an activity of approximately 14.000 MU and an example of a weak promoter is Plac which when induced with IPTG has an activity of approximately 2300 MU. In preferred embodiments, the expression of said nucleic acid sequences are under control of a strong promoter selected from the group consisting of SEQ ID NOs 15, 16, 17, 18, 19, 20, 21, 22, 23, 24, 25 and 26.

In embodiments the expression of said nucleic acid sequences described herein is under control of a PglpF (SEQ ID NO: 27 or Plac (SEQ ID NO: 36) promoter or PmglB_UTR70 (SEQ ID NO: 24) or PglpA_70UTR (SEQ ID NO: 25) or PglpT_70UTR (SEQ ID NO: 26) or variants thereof such as promoters identified in Table 3, in particular the PglpF_SD4 variant of SEQ ID NO: 22 or Plac_70UTR variant of SEQ ID NO: 18, or PmglB_70UTR variants of SEQ ID NO: 15, 16, 17, 19, 20, 21, 23 and 24. Further suitable variants of PglpF, PglpA_70UTR, PglpT_70UTR and PmglB_70UTR promoter sequences are described in or WO2019/123324 and WO2020/255054 respectively (hereby incorporated by reference).

In preferred embodiments, the recombinant nucleic acid sequences individually are under the control of one or more promoters selected from the group consisting of PglpF, Plac, PmglB_70UTR, PglpA_70UTR and PglpT_70UTR (SEQ ID NOs: 27, 36, 24, 25 and 26, respectively) and variants thereof.

Integration of the nucleic acid construct of interest comprised in the construct (expression cassette) into the bacterial genome can be achieved by conventional methods, e.g. by using linear cartridges that contain flanking sequences homologous to a specific site on the chromosome, as described for the attTn7-site (Waddell C. S. and Craig N. L., Genes Dev. (1988) February; 2(2):137-49.); methods for genomic integration of nucleic acid sequences in which recombination is mediated by the Red recombinase function of the phage λ or the RecE/RecT recombinase function of the Rac prophage (Murphy, J Bacteriol. (1998); 180(8):2063-7; Zhang et al., Nature Genetics (1998) 20:123-128 Muyrers et al., EMBO Rep. (2000) 1(3): 239-243); methods based on Red/ET recombination (Wenzel et al., Chem Biol. (2005), 12(3):349-56; Vetcher et al., Appl Environ Microbiol. (2005); 71(4):1829-35); or positive clones, i.e., clones that carry the expression cassette, can be selected e.g., by means of a marker gene, or loss or gain of gene function.

In one or more exemplary embodiments, the present disclosure relates to one or more recombinant nucleic acid sequences as illustrated in SEQ ID NOs: 6, 7, 8, 9 and 10 [nucleic acid encoding Bgall1, Murba1, Bbac1, Para1 and FutA, respectively].

In particular, the present disclosure relates to one or more of a recombinant nucleic acid sequence and/or to a functional homologue thereof having a sequence which is at least 70% identical to SEQ ID NOs: 6, 7, 8, 9 and 10 [nucleic acid encoding Bgall1, Murba1, Bbac1, Para1 and FutA, respectively], such as at least 75% identical, at least 80% identical, at least 85% identical, at least 90% identical, at least, at least 95% identical, at least 98% identical, or 100% identical.

### Sequence Identity

The term “sequence identity” as used herein describes the relatedness between two amino acid sequences or between two nucleotide sequences, i.e., a candidate sequence (e.g., a sequence of the invention) and a reference sequence (such as a prior art sequence) based on their pairwise alignment. For purposes disclosed herein, the sequence identity between two amino acid sequences is determined using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277,), preferably version 5.0.0 or later (available at https://www.ebi.ac.uk/Tools/psa/emboss needle/). The parameters used are gap open penalty of 10, gap extension penalty of 0.5, and the EBLOSUM62 (EMBOSS version of 30 BLOSUM62) substitution matrix. The output of Needle labelled “identity” (obtained using the -nobrief option) is used as the percent identity. Generally sequence identity may be calculated as follows: (Identical Residues×100)/(Length of Aligned region).

For purposes disclosed herein, the sequence identity between two nucleotide sequences is determined using the Needleman-Wunsch algorithm (Needleman and Wunsch, 1 970, supra) as implemented in the Needle program of the EMBOSS package (EMBOSS: The European Molecular Biology Open Software Suite, Rice et al., 2000, Trends Genet. 16:276-277), 10 preferably version 5.0.0 or later. The parameters used are gap open penalty of 10, gap extension penalty of 0.5, and the DNAFULL (EMBOSS version of NCBI NUC4.4) substitution matrix. The output of Needle labelled “identity” (obtained using the -nobrief option) is used as the percent identity. Generally sequence identity may be calculated as follows: (Identical Deoxyribonucleotides×100)/(Length of Aligned region)“ ”.

### Functional Homologue

A functional homologue or functional variant of a protein/nucleic acid sequence as described herein is a protein/nucleic acid sequence with alterations in the genetic code, which retain its original functionality. A functional homologue may be obtained by mutagenesis or may be natural occurring variants from the same or other species. The functional homologue should have a remaining functionality of at least 50%, such as at least 60%, 70%, 80%, 90% or 100% compared to the functionality of the protein/nucleic acid sequence.

A functional homologue of any one of the disclosed amino acid or nucleic acid sequences can also have a higher functionality. A functional homologue of any one of the amino acid sequences shown in table 1 or a recombinant nucleic acid encoding any one of the sequences of SEQ ID NO: 6, 7, 8, 9 and 10, should ideally be able to participate in the production of fucosylated HMOs, in terms of increased HMO yield, export of HMO product out of the cell or import of substrate for the HMO production, such as a acceptor oligosaccharide of at least three monosaccharide units, improved purity/by-product formation, reduction in biomass formation, viability of the genetically engineered cell, robustness of the genetically engineered cell according to the disclosure, or reduction in consumables needed for the production.

### Use of a Genetically Engineered Cell or Enzyme

The disclosure also relates to any commercial use of the enzyme(s), genetically engineered cell(s) or the nucleic acid construct(s) disclosed herein, such as, but not limited to, in a method for producing one or more fucosylated human milk oligosaccharide (HMO), preferably, LNDFH-II.

Accordingly, the present disclosure also relates to the use of a fucosyltransferase with α-1,3(4)-fucosyltransferase activity in production of a fucosylated product comprising LNDFH-II, wherein the fucosyltransferase is selected from the group consisting of Bgall1, Murba1, Bbac1, Para1 and FutA comprising or consisting of an amino acid sequence according to SEQ ID NO: 1, 2, 3, 4 or 5, or a functional homologue thereof with an amino acid sequence that is at least 80% identical to SEQ ID NO: 1, 2, 3, 4 or 5.

In further embodiments, the fucosyltransferase for use in production of a fucosylated product is selected from the group consisting of Bgall1, Murba1 and Bbac1 comprising or consisting of an amino acid sequence according to SEQ ID NO: 1, 2, or 3, or a functional homologue thereof which amino acid sequence is at least 80% identical to SEQ ID NO: 1, 2, or 3.

In embodiments, the fucosyltransferases described herein are also used in the manufacturing of a fucosylated product, wherein the fucosylated product comprises one or more fucosylated oligosaccharides including LNDFH-II.

In an exemplified embodiment, the genetically engineered cell and/or the nucleic acid construct described herein is used in the manufacturing of HMOs. Preferably, in the manufacturing of mixtures of HMOs, wherein the molar % content of LNDFH-II produced by the genetically engineered cell is above 5% of the total amount of HMO produced. Preferably, in the manufacturing of HMOs, wherein a mixture of fucosylated HMOs comprising LNDFH-II is intended as the primary product, the molar % content of LNFP-III produced by the genetically engineered cell is above 5% such as above 10%, such as above 15%, such as above 20%, such as above 25%, such as above 30%, or such as above 35%, of the total amount of HMO produced.

In further embodiments, the fucosyltransferase for use in production of a fucosylated product is selected from the group consisting of Bgall1, Murba1, Bbac1, Para1 comprising or consisting of an amino acid sequence according to SEQ ID NO: 1, 2, 3 or 4, or a functional homologue thereof which amino acid sequence is at least 80% identical to SEQ ID NO: 1, 2, 3 or 4. In embodiments, the fucosyltransferase described herein is also used in the manufacturing of a fucosylated product, wherein the fucosylated product is one or more fucosylated oligosaccharides, such as one or more HMOs, preferably, a mixture of HMOs wherein at least 60%, such as at least 75%, Such as at least 85%, such as at least 90%, such as at least 95% of the mixture consists of LNDFH-II in combination with LNFP-V and/or 3FL.

In an exemplified embodiment, the genetically engineered cell, the α-1,3(4)-fucosyltransferase and/or the nucleic acid construct described herein is used in the manufacturing of one or more fucosylated HMO(s), preferably, LNDFH-II or LNDF-II and LNFP-V.

Production of these HMO's may require the presence of two or more glycosyltransferase activities.

### A Method for Producing Fucosylated Human Milk Oligosaccharides (HMOs)

The present disclosure also relates to a method for producing one or more fucosylated human milk oligosaccharides (HMOs), preferably LNDFH-II, said method comprises culturing a genetically engineered cell described herein.

One aspect is a method for producing one or more fucosylated human milk oligosaccharides (HMOs), said method comprises comprising the steps of:
a) providing a genetically modified cell as described herein; and
b) culturing the cell according to (a) in a suitable cell culture medium to produce said one or more fucosylated HMOs, and
c) optionally, purifying said one or more fucosylated HMOs.

The present disclosure relates to a method for producing one or more fucosylated human milk oligosaccharides (HMOs), said method comprising cultivating a genetically engineered cell, said cell comprising:
a recombinant nucleic acid sequence encoding a fucosyltransferase with α-1,3(4)-fucosyltransferase activity, wherein said fucosyltransferase is selected from the group consisting of:
   a. Bgall1, comprising or consisting of an amino acid sequence according to SEQ ID NO: 1 or a functional homologue thereof with an amino acid sequence that is at least 80% identical to SEQ ID NO: 1, [Bgall1 amino acid];
   b. Murba1 comprising or consisting of an amino acid sequence according to SEQ ID NO: 2 or a functional homologue thereof with an amino acid sequence that is at least 80% identical to SEQ ID NO: 2, [Murba1 amino acid];
   c. Bbac1 comprising or consisting of an amino acid sequence according to SEQ ID NO: 3, or a functional homologue thereof with an amino acid sequence that is at least 80% identical to SEQ ID NO: 3 [Bbac1 amino acid], and
   d. Para1 comprising or consisting of an amino acid sequence according to SEQ ID NO: 4, or a functional homologue thereof with an amino acid sequence that is at least 80% identical to SEQ ID NO: 4 [Para1 amino acid].

The present disclosure also relates to a method for producing one or more fucosylated human milk oligosaccharide (HMO), said method comprising cultivating a genetically engineered cell, said cell comprising:
a recombinant nucleic acid sequence encoding a fucosyltransferase with α-1,3(4)-fucosyltransferase activity, wherein said fucosyltransferase is selected from the group consisting of:
   a. Bgall1, comprising or consisting of an amino acid sequence according to SEQ ID NO: 1, or a functional homologue thereof with an amino acid sequence that is at least 80% identical to SEQ ID NO: 1, [Bgall1 amino acid],
   b. Murba1 comprising or consisting of an amino acid sequence according to SEQ ID NO: 2 or a functional homologue thereof with an amino acid sequence that is at least 80% identical to SEQ ID NO: 2, [Murba1 amino acid],
   c. Bbac1 comprising or consisting of an amino acid sequence according to SEQ ID NO: 3, or a functional homologue thereof with an amino acid sequence that is at least 80% identical to SEQ ID NO: 3 [Bbac1 amino acid] and
   d. Para1 comprising or consisting of an amino acid sequence according to SEQ ID NO: 4, or a functional homologue thereof with an amino acid sequence that is at least 80% identical to SEQ ID NO: 4 [Para1 amino acid], and
wherein at least one of the fucosylated HMOs is LNDFH-II. The fucosylated HMOs produced by the above method may further comprise a fucosylated HMO selected from group consisting of 3FL, LNFP-II and LNFP-V, and potentially also a non-fucosylated HMO such as LNT and/or LNT-II.

A further embodiment relates to a method for producing one or more fucosylated human milk oligosaccharides (HMO), said method comprising cultivating a genetically engineered cell comprising:
a. a recombinant nucleic acid sequence encoding a fucosyltransferase with α-1,3(4)-fucosyltransferase activity, wherein said fucosyltransferase is selected from the group consisting of:
   i. Bgall1, comprising or consisting of an amino acid sequence according to SEQ ID NO: 1, or a functional homologue thereof with an amino acid sequence that is at least 80% identical to SEQ ID NO: 1, [Bgall1 amino acid];
   ii. Murba1 comprising or consisting of an amino acid sequence according to SEQ ID NO: 2, or a functional homologue thereof with an amino acid sequence that is at least 80% identical to SEQ ID NO: 2, [Murba1 amino acid];
   iii. Bbac1 comprising or consisting of an amino acid sequence according to SEQ ID NO: 3, or a functional homologue thereof with an amino acid sequence that is at least 80% identical to SEQ ID NO: 3 [Bbac1 amino acid];
   iv. Para1 comprising or consisting of an amino acid sequence according to SEQ ID NO: 4, or a functional homologue thereof with an amino acid sequence that is at least 80% identical to SEQ ID NO: 4 [Para1 amino acid] and
   v. FutA comprising or consisting of an amino acid sequence according to SEQ ID NO: 5, or a functional homologue thereof with an amino acid sequence that is at least 80% identical to SEQ ID NO: 5 [FutA amino acid]; and
b. a recombinant nucleic acid sequence encoding an enzyme with a β-1,3-galactosyltransferase activity, and
c. optionally, a recombinant nucleic acid sequence encoding an enzyme with β-1,3-N-acetyl-glucosaminyltransferase activity, and
wherein at least one of the fucosylated HMOs is LNDFH-II.

In embodiments the genetically engineered cell is cultured in a suitable medium providing a suitable carbon source and in the presence of an initial substrate selected from lactose or LNT-II. Preferably, the initial substrate is lactose and the cell expresses an enzyme with β-1,3-N-acetyl-glucosaminyltransferase activity.

A further embodiment relates to a method for producing one or more fucosylated human milk oligosaccharides (HMO), said method comprising
a) cultivating a genetically engineered cell comprising
   a. a recombinant nucleic acid sequence encoding a fucosyltransferase with α-1,3 (4)-fucosyltransferase activity, wherein said enzyme is selected from the group consisting of:
      i. Bgall1, comprising or consisting of an amino acid sequence according to SEQ ID NO: 1, or a functional homologue thereof with an amino acid sequence that is at least 80% identical to SEQ ID NO: 1, [Bgall1 amino acid];
      ii. Murba1 comprising or consisting of an amino acid sequence according to SEQ ID NO: 2, or a functional homologue thereof with an amino acid sequence that is at least 80% identical to SEQ ID NO: 2, [Murba1 amino acid] and
      iii. Bbac1 comprising or consisting of an amino acid sequence according to SEQ ID NO: 3, or a functional homologue thereof with an amino acid sequence that is at least 80% identical to SEQ ID NO: 3 [Bbac1 amino acid],
   b. a recombinant nucleic acid sequence encoding an enzyme with a β-1,3-galactosyltransferase activity; and
   c. optionally, a recombinant nucleic acid sequence encoding an enzyme with β-1,3-N-acetyl-glucosaminyltransferase activity; and
b) cultivating said cell in a suitable medium in the presence of an initial substrate, and wherein at least 5% of the molar content of the HMOs produced by the method is LNDFH-II.

A further embodiment relates to a method for producing LNDFH-II and one or more additional fucosylated HMOs, comprising
a) providing a genetically engineered cell capable of producing LNDFH-II, comprising
   a. at least two genomic copies of a recombinant nucleic acid sequence encoding a fucosyltransferase with α-1,3-fucosyltransferase and α-1,4-fucosyltransferase activity, which is capable of fucosylating an oligosaccharide at a GlcNAc moiety and a Glc moiety and wherein the fucosyltransferase is selected from the group consisting of
      i) Bgall1, comprising or consisting of an amino acid sequence according to SEQ ID NO: 1, or a functional homologue thereof with an amino acid sequence that is at least 80% identical to SEQ ID NO: 1 and
      ii) FutA comprising or consisting of an amino acid sequence according to SEQ ID NO: 5, or a functional homologue thereof with an amino acid sequence that is at least 95% identical to SEQ ID NO: 5, and
   b. a recombinant nucleic acid sequence encoding a β-1,3-galactosyltransferase, and
   c. optionally a recombinant nucleic acid sequence(s) encoding β-1,3-N-acetylglucosaminyltransferase, and
b) cultivating said cell in a suitable medium in the presence of an initial substrate and wherein at least 15%, such as at least 20% of the molar content of the HMOs produced by the method is LNDFH-II.

In embodiments the initial substrate is selected from lactose or LNT-II. Preferably, the initial substrate is lactose and the cell expresses an enzyme with β-1,3-N-acetyl-glucosaminyltransferase activity.

In embodiments, the fucosylated HMOs is LNDFH-II. In further embodiments one or more HMOs selected form the groups consisting of 3FL, LNT, LNFP-II and LNFP-V are produced by the method of the invention.

The method particularly comprises cultivating a genetically engineered cell that produces a fucosylated HMO, wherein the LNDFH-II content produced by said cell is at least 5% of the total HMO content produced by the cell. The method particularly comprises culturing a genetically engineered cell that produces a fucosylated HMO, wherein the LNDFH-II and LNFP-V content produced by said method is at least 35% of the total HMO content produced by the cell.

The methods comprising cultivating a genetically engineered cell that produces a fucosylated HMO and further comprises culturing said genetically engineered cell in in the presence of a carbon source (energy source), such as a carbon source selected from the group consisting of glucose, sucrose, fructose, xylose and glycerol.

In one embodiment, the method disclosed herein produces a mixture of HMO(s), wherein at least 50%, such as at least 55%, 60%, 65%, 70%, 75%, 80%, 85%, 90%, 95% or 98% of the molar content of the total amount of HMOs produced is a fucosylated HMO.

In one embodiment, the method disclosed herein produces a mixture of HMO(s), wherein at least 5%, such as at least 6%, 7%, 8%, 9%, 10%, 12%, 15%, 17%, 20%, 25%, 30%, or such as at least 35% of the molar content of the total amount of HMOs produced is LNDFH-II.

In one embodiment, the method disclosed herein produces a mixture of HMO(s), wherein at least 75%, such as at least 80%, 85%, 90%, 95% or at least 97% of the molar content of the total HMOs produced by said method are fucosylated HMOs selected from the group consisting of 3FL, LNFP-II, LNFP-V and LNDFH-II.

In one embodiment, the method disclosed herein produces a mixture of HMO(s), wherein at least 65%, such as at least 70%, 75%, 80%, 85%, 90%, 95% or at least 97% of the molar content of the total HMOs produced by said method are fucosylated HMOs selected from the group consisting of 3FL, LNFP-V and LNDFH-II.

In one embodiment, the method disclosed herein produces a mixture of HMO(s), wherein the produced mixture of HMOs is essentially free of LNFP-II.

In one embodiment, the method disclosed herein produces a mixture of HMO(s), wherein the produced mixture of HMOs is essentially free of 3FL.

In one embodiment, the method disclosed herein produces a mixture of HMO(s), wherein the cell produces a mixture of HMOs comprising 3FL, LNT, LNFP-V and LNDFH-II.

In one embodiment, the method disclosed herein produces a mixture of HMO(s), wherein the cell produces a mixture of HMOs comprising LNT, LNT-II, LNFP-II, LNFP-V and LNDFH-II In one embodiment, the method disclosed herein produces LNDFH-II.

In embodiments the genetically cell used in the method described herein is a genetically engineered cell with one or more of the previously described modifications.

The method disclosed herein comprises providing a glycosyl donor, which is synthesized separately by one or more genetically engineered cells and/or is exogenously added to the culture medium from an alternative source. Preferably the glucosyl donor is produced by an endogenous or recombinant de novo pathway in the genetically engineered cell.

One embodiment, disclosed herein further comprises providing an acceptor saccharide as initial substrate for the HMO formation, the acceptor saccharide comprising at least two monosaccharide units, which is exogenously added to the culture medium and/or has been produced by a separate microbial fermentation. As an alternative to adding the initial substrate for the production of the HMO to the fermentation medium, the genetically modified cell may be further engineered to produce the initial substrate inside the cell (see for example WO2015/150328).

In one embodiment, the method disclosed herein comprises providing an acceptor saccharide comprising at least two monosaccharide units, which is selected form lactose, LNT-II and LNT, and added prior to and/or during the cultivation of the genetically modified cell. In a preferred embodiment the initial substrate for HMO formation is lactose which is fed to the culture during the fermentation of the genetically engineered cell.

The fucosylated human milk oligosaccharide (HMO) is retrieved from the culture, either from the culture medium and/or the genetically engineered cell.

### Culturing/Fermenting

Culturing, cultivation, fermenting or fermentation (used interchangeably herein) in a controlled bioreactor typically comprises (a) a first phase of exponential cell growth in a culture medium ensured by a carbon-source, and (b) a second phase of cell growth in a culture medium run under carbon limitation, where the carbon-source is added continuously together with the acceptor oligosaccharide, such as lactose, allowing formation of the HMO product in this phase. By carbon (sugar) limitation is meant the stage in the fermentation where the growth rate is kinetically controlled by the concentration of the carbon source (sugar) in the culture broth, which in turn is determined by the rate of carbon addition (sugar feed-rate) to the fermenter.

The terms “manufacturing” or “manufacturing scale” or “large-scale production” or “large-scale fermentation”, are used interchangeably and in the meaning of the invention defines a fermentation with a minimum volume of 100 L, such as 1000 L, such as 10.000 L, such as 100.000 L, such as 200.000 L culture broth. Usually, a “manufacturing scale” process is defined by being capable of processing large volumes yielding amounts of the HMO product of interest that meet, e.g., in the case of a therapeutic compound or composition, the demands for toxicity tests, clinical trials as well as for market supply. In addition to the large volume, a manufacturing scale method, as opposed to simple lab scale methods like shake flask cultivation, is characterized by the use of the technical system of a bioreactor (fermenter) which is equipped with devices for agitation, aeration, nutrient feeding, monitoring and control of process parameters (pH, temperature, dissolved oxygen tension, back pressure, etc.). To a large extent, the behaviour of an expression system in a lab scale method, such as shake flasks, benchtop bioreactors or the deep well format described in the examples of the disclosure, does allow to predict the behaviour of that system in the complex environment of a bioreactor.

With regards to the suitable cell medium used in the fermentation process, there are no limitations. The culture medium may be semi-defined, i.e., containing complex media compounds (e.g., yeast extract, soy peptone, casamino acids, etc.), or it may be chemically defined, without any complex compounds. The carbon-source can be selected from the group consisting of glucose, sucrose, fructose, xylose and glycerol. In one or more exemplary embodiments, the culturing media is supplemented with one or more energy and carbon sources selected form the group containing glycerol, sucrose and glucose. In additional embodiments, lactose is added during the cultivation of the genetically engineered cells as a substrate for the HMO formation.

In one or more exemplary embodiments, the culturing media contains sucrose as the sole carbon and energy source. In one or more exemplary embodiments, the genetically engineered cell comprises one or more heterologous nucleic acid sequence encoding one or more heterologous polypeptide(s) which enables utilization of sucrose as sole carbon and energy source of said genetically engineered cell.

In one or more exemplary embodiments, the genetically engineered cell comprises a PTS-dependent sucrose utilization system, further comprising the scrYA and scrBR operons as described in WO2015/197082 (hereby incorporated by reference).

After carrying out the method of this invention, the fucosylated HMO produced can be collected from the cell culture or fermentation broth in a conventional manner.

### Retrieving/Harvesting

The fucosylated human milk oligosaccharide (HMO) is retrieved from the culture medium and/or the genetically engineered cell. In the present context, the term “retrieving” is used interchangeably with the term “harvesting”. Both “retrieving” and “harvesting” in the context relate to collecting the produced HMO(s) from the culture/broth following the termination of fermentation. In one or more exemplary embodiments it may include collecting the HMO(s) included in both the biomass (i.e., the host cells) and cultivation media, i.e., before/without separation of the fermentation broth from the biomass. In other embodiments, the produced HMOs may be collected separately from the biomass and fermentation broth, i.e., after/following the separation of biomass from cultivation media (i.e., fermentation broth).

The separation of cells from the medium can be carried out with any of the methods well known to the skilled person in the art, such as any suitable type of centrifugation or filtration. The separation of cells from the medium can follow immediately after harvesting the fermentation broth or be carried out at a later stage after storing the fermentation broth at appropriate conditions. Recovery of the produced HMO(s) from the remaining biomass (or total fermentation broth) include extraction thereof from the biomass (i.e., the production cells).

After recovery from fermentation, HMO(s) are available for further processing and purification.

The HMOs can be purified according to the procedures known in the art, e.g., such as described in WO2017/152918, WO2017/182965 or WO2015/188834, wherein the latter describes purification of fucosylated HMOs. The purified HMOs can be used as nutraceuticals, pharmaceuticals, or for any other purpose, e.g., for research.

At the end of culturing, the oligosaccharide as product can be accumulated both in the intra- and the extracellular matrix.

The method described herein comprises cultivating the genetically engineered microbial cell in a culture medium which is designed to support the growth of microorganisms, and which contains one or more carbohydrate sources or just carbon-source, such as selected from the group consisting of glucose, sucrose, fructose, xylose and glycerol. In one or more exemplary embodiments, the culturing media is supplemented with one or more energy and carbon sources selected form the group containing glycerol, sucrose and glucose.

### Manufactured Product

The term “manufactured product” refers to the one or more HMOs intended as the one or more product HMO(s) or composition of a mixture of HMOs. Preferably, the product HMOs or composition is produced by a method described herein using a genetically engineered cell described herein.

One embodiment relates to a composition of HMOs consisting essentially of LNDFH-II, LNFP-V, LNT and 3FL with low amounts of LNT-II and LNFP-II, such as below 10% total molar HMO content in the composition.

Another embodiment relates to a composition of HMOs consisting essentially of 15-30 molar % of LNDFH-II and 20-60 molar % LNFP-V, 0-15 molar % LNT and 15-60% 3FL, in total adding up to 100% molar content.

Another embodiment relates to a composition of HMOs consisting essentially of 1-10 molar % of LNDFH-II and 35-70 molar % LNFP-V, 0-25 molar % LNT and 5-55% 3FL, in total adding up to 100% molar content.

Another embodiment relates to a composition of HMOs consisting essentially of 2-12 molar % of LNDFH-II and 62-72 molar % LNFP-V, 0-10 molar % LNT and 17-27% 3FL, in total adding up to 100% molar content.

Another embodiment relates to a composition of HMOs consisting essentially of 1-15 molar % of LNDFH-II and 2-10 molar % LNFP-V, 2-10 molar % LNFP-II, 30-55 molar % LNT and 35-50% LNT-II, in total adding up to 100% molar content.

Another embodiment relates to a composition of HMOs consisting essentially of 2-40 molar % of LNDFH-II and 40-82 molar % LNFP-V, 2-25 molar % LNT and 0-20% 3FL, in total adding up to 100% molar content.

Advantageously, the methods disclosed herein provide valuable mixtures of HMOs with high levels of fucosylated HMOs including the complex HMO LNDFH-II. Some of the genetically engineered cells described herein produce sufficient LNDFH-II, such as above 20% of the total HMO, to facilitate it purification from the mixture of HMOs produced by the cultivation.

The manufactured product may be a powder, a composition, a suspension, or a gel comprising one or more HMOs.

### Use of Compositions of or Mixtures of HMOs

Naturally occurring in breast milk, HMOs have evolved over thousands of years, with HMO research (clinical and preclinical) now suggesting that specific HMOs at the correct level of supplementation can provide us with unique health benefits. As fucosylated HMOs constitutes more than 60%, such as more than 75%, such as more than 85%, such as more than 90%, such as more than 95% of the total HMOs in human milk, mixtures with a high content of fucosylated HMOs are more natural.

Accordingly, LNDFH-II and mixtures of HMOs comprising LNDFH-II are highly relevant as either a nutritional supplement or as a therapeutic.

Clinical data in infants indicate that Human Milk Oligosaccharide supplements may help to develop the desired microbiota by serving as a food source for the beneficial bacteria in the intestine. In particular, Human Milk Oligosaccharide supplements may help support immunity and gut health, with a potential role in cognitive development, which may open future innovation opportunities.

The mixtures or composition of HMOs may be used to enhance the beneficial bacteria in the gut microbiome. Beneficial bacteria are for example bacteria of the Bifidobacterium sp., Lactobacillus sp. or Barnesiella sp. The enhancement of beneficial bacteria may in turn lead to increased production of short chain fatty acids (SCFAs) such as acetate, propionate and butyrate which have been shown to have many benefits in infants and young children, such as inhibition of pathogen bacteria, prevention of infection and diarrhoea, reduced risk of allergy and metabolic disorders (see for example WO2006/130205, WO 2017/129644, WO2017/129649).

The mixtures or composition of HMOs produced according to the method described herein may be used to reduce the abundance of undesirable viruses and bacteria in the gut microbiome. Examples of pathogenic bacteria and viruses that may be reduced by the HMO mixtures described herein are including Candida albicans, Clostridium difficile, Enterococcus faecium, Escherichia coli, Helicobacter pylori, Streptococcus agalactiae, Shigella dysenteriae, Staphylococcus aureus, nora virus and rota virus. Each composition described herein can also be used to treat and/or reduce the risk of a broad range of bacterial infections of a human.

Probiotics may be consumed as live bacteria or as a dried (e.g. lyophilized) product. Independent of the drying method, rehydration involves an important step in the recovery of dehydrated bacteria; an inadequate rehydration/regeneration step may lead to poor cell viability and a low final survival rate. Rehydration is therefore a highly critical step in the revitalization of a lyophilized culture. For both live and rehydrated bacteria, the survival of the bacteria under acidic conditions is critical since they need to pass through the acidic environment of the stomach and may also be faced with storage (shelf-life) in acidic food products.

The mixtures or composition of HMOs produced according to the method described herein may be used to increase the regeneration and viability of lyophilized probiotics, including probiotics of Bifidobacterium sp, and/or Lactobacillus sp. Examples of Bifidobacterium sp which may have increased regeneration and viability are Bifidobacterium animals lactis BB12 DSM 32269, Bifidobacterium animals lactis BIF6, Bifidobacterium longum DSM 32946, Bifidobacterium longum BB536, Bifidobacterium bifidum DSMZ 32403, Bifidobacterium infantis, Bifidobacterium breve DSM 33789, Bifidobacterium infantis SP37 DSM 32687, Bifidobacterium adolescentis DSM 34065 and/or Bifidobacterium animalis ssp. animalis DSM 16284. Examples of Lactobacillus sp which may have increased regeneration and viability are Lactobacillus rhamnosus GG DSM 32550, Lactobacillus rhamnosus 19070-2 DSM 26357, Lactobacillus rhamnosus GG, Lactobacillus rhamnosus LBrGG, Lactobacillus reuteri DSM 12246, Lactobacillus plantarum TIFN101, Lactobacillus gasseri Lg-36 200B FloraFit Danisco, Lactobacillus casei DSM 32382, Lactobacillus paracasei, Lactobacillus plantarum PS 128, Lactobacillus plantarum (Sacco) DSM 32383, Lactococcus lactis PAREVE, Lactobacillus paracasei ssp. Paracasei and/or Lactobacillus Probio-Tec® LGG®, Limosilactobacillus reuteri S12 DSM 33752.

In the context of the present application “Regeneration” means the process of regaining/restoring a dried bacteria's viability (i.e., “reviving” the bacterial cells by rehydration, wherein “rehydration” means restoring fluid). This process is also sometimes referred to as “reconstitution”.

In the context of the present application “Viability” is the ability of a bacterial cell to live and function as a living cell. One way of determining the viability of bacterial cells is by spreading them on an agar plate with suitable growth medium and counting the number of colonies formed after incubation for a predefined time (plate counting). Alternatively, FACS analysis may be used.

In the context of the present application “Improving the regeneration” of Bifidobacterium sp and/or Lactobacillus sp bacteria means to increase the amount (number) of Bifidobacterium sp and/or Lactobacillus sp. bacteria successfully regenerating/reviving compared to the respective control (i.e., the amount/number of Bifidobacterium sp and/or Lactobacillus sp. bacteria without the addition of HMO).

In the context of the present application “Improving the viability” of Bifidobacterium sp and/or Lactobacillus sp bacteria means to increase the amount (number) of viable Bifidobacterium sp and/or Lactobacillus sp. bacteria compared to the respective control (i.e., the amount/number of Bifidobacterium sp and/or Lactobacillus sp. bacteria without the addition of HMO).

The mixtures or composition of HMOs produced according to the method described herein, may be used to extend the shelf life of probiotics, such as Bifidobacterium sp, and/or Lactobacillus sp.

An embodiment of the present invention is a composition comprising a mixture of HMOs as described herein (in particular in the section “Mixtures of HMOs” and one or more probiotics. Preferably, the probiotic is a Bifidobacterium sp and/or Lactobacillus sp such as any of the specific species mentioned above.

The mixtures or composition of HMOs produced according to the method described herein, may be used to improve the flowability of a powder or decrease the viscosity of a liquid.

Composition and mixtures of HMOs described in the section “Manufactured product” may also form part of a composition comprising additional parts, such as active pharmaceutical ingredients, food supplements, excipients, surfactants etc.

Accordingly, embodiments relate to the use of a composition or a mixture of HMOs as described herein as a dietary supplement or medical nutrition. In further embodiments the composition for use in an infant formula, a dietary supplement or medical nutrition comprises LNDFH-II, 3FL and LNFP-V.

The mixtures or composition of HMOs described herein are used in a nutritional composition. Nutritional compositions are for example, an infant formula, a rehydration solution, or a dietary maintenance, medical nutrition or supplement for elderly individuals or immunocompromised individuals. Macronutrients such as edible fats, carbohydrates and proteins can also be included in such anti-infective compositions. Edible fats include, for example, coconut oil, soy oil and monoglycerides and diglycerides. Carbohydrates include, for example, glucose, edible lactose and hydrolysed cornstarch. Proteins include, for example, soy protein, whey, and skim milk. Vitamins and minerals (e. g. calcium, phosphorus, potassium, sodium, chloride, magnesium, manganese, iron, copper, zinc, selenium, iodine, and Vitamins A, E, D, C, and B complex) can also be included in such anti-infective compositions.

In embodiments, the composition comprising a mixture of HMOs produced as described herein is a pharmaceutical composition.

The present invention also relates to the use of a mixture or composition as described herein as a dietary supplement and/or medical nutrition.

In embodiments, the invention relates to the use of a mixture or composition described herein in infant nutrition.

### Sequences

The current application contains a sequence listing in text format and electronical format which is hereby incorporated by reference.

An overview of the SEQ ID NOs used in the present application can be found in table 1 (α-1,3(4)-fucosyltransferase protein sequences) and table 3 (promoter sequences), additional sequences described in the application is the DNA sequences encoding the α-1,3(4)-fucosyltransferases (SEQ ID NO: 6 to 10), the DNA sequence encoding the colanic acid gene cluster from E. coli (SEQ ID NO: 11) and the β-1,3-N-acetylglucosaminyltransferase LgtA from N. meningitidis (SEQ ID NO: 12), β-1,3-galactosyltransferases galTK from H. pylori (SEQ ID NO: 13), Lactose permease, LacY (SEQ ID NO: 14), FucTIII α-1,3-fucosyltransferase (SEQ ID NO: 39) and the DNA sequence encoding it (SEQ ID NO: 40).

### Items

1. A genetically engineered cell capable of producing LNDFH-II, comprising a recombinant nucleic acid sequence encoding a fucosyltransferase, wherein the glycosyltransferase is selected from the group consisting of,
a. Bgall1 comprising or consisting of an amino acid sequence according to SEQ ID NO: 1, or a functional homologue thereof with an amino acid sequence that is at least 80% identical to SEQ ID NO: 1,
b. Murba1 comprising or consisting of an amino acid sequence according to SEQ ID NO: 2, or a functional homologue thereof with an amino acid sequence that is at least 80% identical to SEQ ID NO: 2,
c. Bbac1 comprising or consisting of an amino acid sequence according to SEQ ID NO: 3, or a functional homologue thereof with an amino acid sequence that is at least 80% identical to SEQ ID NO: 3, and
d. Para1 comprising or consisting of an amino acid sequence according to SEQ ID NO: 4, or a functional homologue thereof with an amino acid sequence that is at least 80% identical to SEQ ID NO: 4.

2. The genetically engineered cell according to item 1, wherein the fucosyltransferase has α-1,3(4)-fucosyltransferase activity

3. The genetically engineered cell according to item 1 or 2, wherein the fucosyltransferase is capable of fucosylating the N-acetylglucosamine (GlcNAc) moiety and the glucose (Glc) moiety in an acceptor oligosaccharide.

4. The genetically engineered cell according to item 1 to 3, wherein the fucosyltransferase exhibit low or no specificity for the galactose moiety in LNT as acceptor oligosaccharide.

5. The genetically engineered cell according to item 3 or 4, wherein the acceptor oligosaccharide for the α-1,3(4)-fucosyltransferase is selected from LNT, LNFP-V and LNFP-II.

6. The genetically engineered cell according to any of the preceding items, wherein the genetically engineered cell comprises one or more further recombinant nucleic acids encoding one or more heterologous glycosyltransferases

7. The genetically engineered cell according to any one of the preceding items, wherein the cell further comprises a recombinant nucleic acid sequence encoding a β-1,3-galactosyltransferase.

8. The genetically engineered cell according to item 6 or 7, wherein the genetically engineered cell further comprises a recombinant nucleic acid sequence encoding a β-1,3-N-acetyl-glucosaminyltransferase.

9. The genetically engineered cell according to any one of items 7 or 8, wherein the β-1,3-N-acetylglucosaminyltransferase is from Neisseria meningitidis and the β-1,3-galactosyltransferase is from Helicobacter pylori.

10. The genetically engineered cell according to item 6 to 9, wherein the 1,3-N-acetylglucosaminyltransferase has an amino acid sequence according to SEQ ID NO: 12, or a functional homologue thereof with an amino acid sequence that is at least 80% identical to SEQ ID NO: 12 and the β-1,3-galactosyltransferase is has an amino acid sequence according to SEQ ID NO: 13, or a functional homologue thereof with an amino acid sequence that is at least 80% identical to SEQ ID NO: 13.

11. The genetically engineered cell according to any one of the preceding, wherein the cell further comprises a substrate importer selected from a lactose importer, a lacto-N-triose-II (LNT-II) importer or an LNT importer.

12. The genetically engineered cell according to any of the preceding items, wherein the cell further produces more than 1 molar % LNDFH-II, preferably more than 5 molar %, preferably more than 10 molar %, preferably more than 15 molar %, preferably more than 20 molar % LNDFH-II of the total amount of HMO produced by the cell.

13. The genetically engineered cell according to any of the preceding items, wherein the cell further produces one or more HMOs selected from the group consisting of 3FL, LNT-II, LNT, pLNH2, LNFP-II and LNFP-V.

14. The genetically engineered cell according to any of the preceding items, wherein the cell produces a mixture of fucosylated HMOs comprising or consisting of LNFP-V and LNDFH-II.

15. The genetically engineered cell according to any of the preceding items, wherein the cell further produces a mixture of HMOs comprising or consisting essentially of
a. LNDFH-II, LNFP-V and LNT, or
b. LNDFH-II, LNFP-V and 3FL, or
c. LNDFH-II, LNFP-V, LNT and 3FL, or
d. LNDFH-II, LNT-II, LNFP-V, LNFP-II, and LNT.

16. The genetically engineered cell according to any of the preceding items wherein the cell produces more than 75 molar %, such as more than 85 molar %, such as more than 90 molar %, such as more than 95 molar % fucosylated HMOs of the total HMO produced by the cell.

17. The genetically engineered cell according to any of the preceding items wherein the cell produces a mixture of 3FL, LNFP-V and LNDFH-II wherein the mixture constitutes at least 90 molar % of the total HMO produced by the cell.

18. The genetically engineered cell according to any of the preceding items, wherein the recombinant nucleic acid sequences individually are under the control of one or more promoters selected from the group consisting of PglpF, Plac, PmglB_70UTR, PglpA_70UTR and PglpT_70UTR (SEQ ID NOS: 27, 36, 24, 25 and 26) and variants thereof as disclosed in table 3.

19. The genetically engineered cell according to item 18, wherein the promoter is a strong promoter selected from the group consisting of SEQ ID NOs 15, 16, 17, 18, 19, 20, 21, 22, 23, 24, 25, 26 and 27.

20. The genetically engineered cell according to any of the preceding items, wherein the cell comprises two, three or more genomic copies and/or a plasmid-borne copy of the recombinant nucleic acid sequence encoding the glycosyltransferase is selected from the group consisting of Bgall1, Murba1 Bbac1 and Para1 comprising or consisting of an amino acid sequence according to SEQ ID NO: 1, 2, 3 or 4, or a functional homologue thereof with an amino acid sequence that is at least 80% identical to SEQ ID NO: 1, 2, 3, or 4.

21. The genetically engineered cell according to item 20, wherein the cell comprises two, three or more genomic copies of Bgall1, Murba1, bac1 or Para1.

22. The genetically engineered cell according to item 20, wherein the cell comprises a high copy number plasmid encoding Bgall1, Murba1, bac1 or Para1.

23. The genetically engineered cell according to any of the preceding items, wherein the cell overexpresses at least one enzyme in the de novo GDP-fucose pathway responsible for the formation of GDP-fucose.

24. The genetically engineered cell according to item 23, wherein the one or more enzyme is selected from the group consisting of mannose-6 phosphate isomerase (manA), phosphomannomutase (manB), mannose-1-phosphate guanylyltransferase guanylyltransferase (manC), GDP-mannose-4,6-dehydratase (gmd) and GDP-L-fucose synthase (wcaG).

25. The genetically engineered cell according to any of the preceding items, wherein the cell comprises a recombinant nucleic acid sequence according to SEQ ID NO: 11 encoding the colanic acid (CA) gene cluster.

26. The genetically engineered cell according to any of the preceding items, wherein said engineered cell is a microorganism.

27. The genetically engineered cell according to any of the preceding items, wherein said engineered cell is a bacterium or a fungus.

28. The genetically engineered cell according to item 27, wherein said fungus is selected from a yeast cell of the genera Komagataella, Kluyveromyces, Yarrowia, Pichia, Saccaromyces, Schizosaccharomyces or Hansenula or from a filamentous fungus of the genera Aspergillus, Fusarium or Trichoderma.

29. The genetically engineered cell according to item 27, wherein said bacterium is selected from the group consisting of Escherichia sp., Bacillus sp., Lactobacillus sp., Corynebacterium sp. and Campylobacter sp.

30. The genetically engineered according to any of item 26 to 29 wherein said engineered cell is selected from the group consisting of Escherichia Coli, Bacillus subtilis, Lactobacillus lactis, Corynebacterium glutamicum Yarrowia lipolytica, Pichia pastoris, and Saccharomyces cerevisiae.

31. The genetically engineered cell according to item 29 or 30, wherein said engineered cell is a microorganism is E. coli.

32. A method for producing one or more fucosylated HMOs, said method comprising cultivating a genetically engineered cell comprising a recombinant nucleic acid sequence encoding a fucosyltransferase with α-1,3(4)-fucosyltransferase activity, wherein said fucosyltransferase is selected from the group consisting of:
a. Bgall1 comprising or consisting of an amino acid sequence according to SEQ ID NO: 1, or a functional homologue thereof with an amino acid sequence that is at least 80% identical to SEQ ID NO: 1,
b. Murba1 comprising or consisting of an amino acid sequence according to SEQ ID NO: 2, or a functional homologue thereof with an amino acid sequence that is at least 80% identical to SEQ ID NO: 2,
c. Bbac1 comprising or consisting of an amino acid sequence according to SEQ ID NO: 3, or a functional homologue thereof with an amino acid sequence that is at least 80% identical to SEQ ID NO: 3,
d. Para1 comprising or consisting of an amino acid sequence according to SEQ ID NO: 4, or a functional homologue thereof with an amino acid sequence that is at least 80% identical to SEQ ID NO: 4 and
e. FutA comprising or consisting of an amino acid sequence according to SEQ ID NO: 5, or a functional homologue thereof with an amino acid sequence that is at least 80% identical to SEQ ID NO: 5, and wherein at least one of the fucosylated HMOs is LNDFH-II.

33. The method according to item 32, wherein at least 5% of the molar content of the total HMOs produced by the genetically engineered cell cultivated in said method is LNDFH-II.

34. The method according to item 32 or 33, wherein at least 65%, such as at least 70%, 80%, 90%, 95% or at least 97% of the molar content of the total HMOs produced by said method are fucosylated HMOs selected from the group consisting of 3FL, LNFP-V and LNDFH-II.

35. The method according to any of items 32 to 34, wherein the genetically engineered cell is a cell according to any one of items 1 to 31.

36. The method according to item 35, wherein the method comprises cultivating the genetically engineered cell in the presence of an energy source selected from the group consisting of glucose, sucrose, fructose, xylose and glycerol.

37. The method according to any one of items 32 to 36, wherein the genetically engineered cell is cultivated in the presence of an acceptor substrate selected from lactose, LNT-II or LNT.

38. The method according to item 32 to 37, wherein the fucosylated HMO(s) produced by the method is purified to at least 95% purity.

39. Use of a fucosyltransferase with α-1,3/4-fucosyltransferase activity in the production of one or more fucosylated HMOs, wherein the enzyme is selected from the group consisting of Bgall1, Murba1, Bbac1, Para1 and FutA comprising or consisting of an amino acid sequence according to SEQ ID NO: 1, 2, 3, 4, or 5 or a functional homologue thereof with an amino acid sequence that is at least 80% identical to SEQ ID NO: 1, 2, 3, 4, or 5, wherein one of the HMOs produced is LNDFH-II.

40. A mixture of HMOs produced with a method according to any of items 32 to 38, consisting essentially of
a. LNDFH-II, LNFP-V and LNT, or
b. LNDFH-II, LNFP-V and 3FL, or
c. LNDFH-II, LNFP-V, LNT and 3FL, or
d. LNDFH-II, LNT-II, LNFP-V, LNFP-II, and LNT.

41. A composition of HMOs consisting essentially of 1-40 molar % of LNDFH-II, 2-85 molar % LNFP-V, 0-10 molar % LNFP-II, 0-65 molar % 3FL, 0-55 molar % LNT and 0-50 molar % LNT-II, in total adding up to 100% molar content.

42. The composition according to item 41, wherein the mixture is selected from the group consisting of:
a. a mixture consisting essentially of 15-40 molar % of LNDFH-II and 20-60 molar % LNFP-V, 0-15 molar % LNT and 15-60% 3FL, in total adding up to 100% molar content;
b. a mixture consisting essentially of 15-40 molar % of LNDFH-II and 15-40 molar % LNFP-V, 0-5 molar % LNT and 25-60% 3FL, in total adding up to 100% molar content;
c. a mixture consisting essentially of 1-15 molar % of LNDFH-II and 35-70 molar % LNFP-V, 0-25 molar % LNT and 5-55% 3FL, in total adding up to 100% molar content;
d. a mixture consisting essentially of 2-15 molar % of LNDFH-II and 60-75 molar % LNFP-V, 0-10 molar % LNT and 15-30% 3FL, in total adding up to 100% molar content; and
e. a mixture consisting essentially of 2-15 molar % of LNDFH-II and 0-10 molar % LNFP-V, 0-10 molar % LNFP-II, 30-50 molar % LNT-II, 30-60 molar % LNT and 0-5 molar % 3FL, in total adding up to 100% molar content, and
f. a mixture consisting essentially of 2-40 molar % of LNDFH-II and 35-85 molar % LNFP-V, 0-25 molar % LNT and 0-20% 3FL, in total adding up to 100% molar content.

43. Use of a mixture or composition of HMOs according to item 40 to 42, in an infant formula, a dietary supplement and/or medical nutrition.

### EXAMPLES

### Methods

Unless stated otherwise, standard techniques, vectors, control sequence elements, and other expression system elements known in the field of molecular biology are used for nucleic acid manipulation, transformation, and expression. Such standard techniques, vectors, and elements can be found, e.g., in: Ausubel et al. (eds.), Current Protocols in Molecular Biology (1995) (John Wiley & Sons); Sambrook, Fritsch, & Maniatis (eds.), Molecular Cloning (1989) (Cold Spring Harbor Laboratory Press, NY); Berger & Kimmel, Methods in Enzymology 152: Guide to Molecular Cloning Techniques (1987) (Academic Press); Bukhari et al. (eds.), DNA Insertion Elements, Plasmids and Episomes (1977) (Cold Spring Harbor Laboratory Press, NY); Miller, J. H. Experiments in molecular genetics (1972.) (Cold spring Harbor Laboratory Press, NY)

The embodiments described below are selected to illustrate the invention and are not limiting the invention in any way.

### Enzymes:

Screening of 50 enzymes with fucosyltransferase activity provided 3 enzymes surprisingly showed dual fucosyltransferase activity, with an α-1,3-fucosyltransferase activity on the Glucose and α-1,4-fucosyltransferase activity on N-Acetylglucosamine moiety of LNT, resulting in production of the complex di-fucosylated HMO LNDFH-II. The GenBank ID and origin of the eight fucosyltransferases are provided in Table 4.

TABLE 4
List of the enzymes tested in the
framework of the present disclosure

| Enzyme | | SEQ | |
|---|---|---|---|
| Name | GenBank ID\hat{ } | ID NO: | Origin |
| Bgall1* | WP_204430034.1 | 1 | Bacteroides gallinaceum |
| Murba1 | MBP3303338.1 | 2 | Muribaculaceae bacterium |
| Bbac1 | MBR5851405.1 | 3 | Bacteroidaceae bacterium |
| Para1 | WP_147381213.1 | 4 | Parabacteroides sp. |
| | | | AM08-6 |
| FutA** | WP_064780098.1 | 5 | Helicobacter pylori ATCC |
| | | | 26695 |
| FucTIII*** | AAR88243 | 39 | Helicobacter pylori DSM |
| | | | 6709 |

\hat{ }the sequences used in the present application may be truncated at the N- or C-terminal as compared to the GenBank sequence these are represented by the SEQ ID NO.
*Bgall1 (FutM2) has been shown to produce 3FL in Chen et al., 2022 Arg Food Chem, and CN114107152.
**FutA has been shown to produce LNFP-V in WO2020115671.
***FucTIII has been shown to produce LNDFH-II in Baumgärtner et al 2015 (Bioorganic & Medicinal Chemistry 23: 6799-6806)

### Strains

The strains (genetically engineered cells) constructed in the present application were based on Escherichia coli K-12 DH1 with the genotype: F⁻, λ⁻, gyrA96, recA1, relA1, endA1, thi-1, hsdR17, supE44. Additional modifications were made to the E. coli K-12 DH1 strain to generate the MDO strain with the following modifications: lacZ: deletion of 1.5 kbp, lacA: deletion of 0.5 kbp, nanKETA: deletion of 3.3 kbp, melA: deletion of 0.9 kbp, wcaJ: deletion of 0.5 kbp, mdoH: deletion of 0.5 kbp, and insertion of Plac promoter upstream of the gmd gene.

Methods of inserting gene(s) of interest into the genome of E. coli are well known to the person skilled in the art. Insertion of genetic cassettes into the E. coli chromosome can be done using gene gorging (see e.g., Herring and Blattner 2004 J. Bacteriol. 186:2673-81 and Warming et al 2005 Nucleic Acids Res. 33(4): e36) with specific selection marker genes and screening methods.

To obtain an LNT producing strain the MDO strain was further engineered by chromosomally integrating a β-1,3-GlcNAc transferase (LgtA from Neisseria meningitidis, homologous to NCBI Accession nr. WP_033911473.1 and as shown in SEQ ID NO: 12) and a β-1,3-galactosyltransferase (GalTK from Helicobacter pylori, homologous to GenBank Accession nr. BD182026.1 and as shown in SEQ ID NO: 13) both under the control of a PglpF promoter (SEQ ID NO: 27), this strain is named the LNT strain.

Codon optimized DNA sequences encoding individual α-1,3/4-fucosyltransferases were genomically integrated into the LNT strain.

The genotypes of the background strain (MDO), the LNT strain and the α-1,3/4-fucosyltransferase expressing strains capable of producing LNDFH-II, and mixtures thereof are provided in Table 5.

TABLE 5
Genotypes of the strains, capable of producing
LNNDFH-II, used in the present examples.

| | | α-1,3FT* cDNA |
|---|---|---|
| Strain ref | Genotype | SEQ ID NO |
| MDO | F−λ− ΔendA1 ΔrecA1 ΔrelA1 ΔgyrA96 Δthi-1 glnV44 | — |
| | hsdR17(rK−mK−) ΔlacZ wcaF::Plac ΔnanKETA ΔlacA | |
| | ΔmelA ΔwcaJ ΔmdoH | |
| LNT | MDO, 2x lgtA-PglpF¹, 1x galTK-PglpF² | — |
| Bgall1-1 | LNT, Bgall1-PglpF, CA-PglpF³ | 6 |
| Bgall1-2 | LNT, 2x Bgall1-PglpF, CA-PglpF³ | 6 |
| Bgall1-3 | LNT, 2x Bgall1-PglpF, pUC57-Bgall1-PglpF⁶, CA-PglpF³ | 6 |
| Murba1-1 | LNT, Murba1-PglpF, CA-PglpF³ | 7 |
| Murba1-2 | LNT, 2x Murba1-PglpF, CA-PglpF³ | 7 |
| Murba1-3 | LNT, 2x Murba1-PglpF, pUC57-Murba1-PglpF⁴, CA- | 7 |
| | PglpF³ | |
| Bbac1 | LNn, Bbac1-PglpF, CA-PglpF³ | 8 |
| Para1-1 | LNT, Para1-PglpF, CA-PglpF³ | 9 |
| Para1-2 | LNT, 2x Para1-PglpF, CA-PglpF³ | 9 |
| FutA-1 | LNT, FutA-PglpF⁴, CA-PglpF³ | 10 |
| FutA-2 | LNT, 2x FutA-PglpF⁴, CA-PglpF³ | 10 |
| FutA-3 | LNT, 2x FutA-PglpF⁴, pUC57-FutA-PglpF⁴, CA-PglpF³ | 10 |
| FucT-III | LNT, FucTIII-PglpF⁵, CA-PglpF³ | 40 |

*1,3FT is an abbreviation of α-1,3-fucosyltransferase, and the DNA sequence is inserted into the genome of the host strain.
¹lgtA-PglpF - two genomically inserted copies of a gene encoding β-1,3-N-acetyl-glucosaminyltransferase (SEQ ID NO: 12) under control of a PglpF promoter.
²galTK-PglpF - one genomically inserted gene encoding β-1,3-Galactosyltransferase (SEQ ID NO: 13) under control of a PglpF promoter (SEQ ID NO: 27).
³CA = extra colanic acid gene cluster (gmd-wcaG-wcaH-wcal-manC-manB, SEQ ID NO: 11) under the control of a PglpF promoter at a locus that is different than the native locus.
⁴FutA-PglpF- gene encoding α-1,3(4)-fucosyltransferase (SEQ ID NO: 5) under control of a PglpF promoter (SEQ ID NO: 27). Either one (1x), two (2x), or three (3x) genomically inserted copies or expression from plasmid pUC57, which is high-copy number (>300) plasmid having the ColE1/pMB1/pBR322/pUC origin of replication. The antibiotic resistance marker on the pUC57 vector is ampicillin.
⁵FucT-III-PglpF - single copy of gene encoding α-1,4-fucosyltransferase (SEQ ID NO: 39) under control of a PglpF promoter (SEQ ID NO: 27). Disclosed in Baumgärtner et al 2015 Bioorganic & Medicinal Chemistry 23: 6799-6806

### Deep Well Assay

Deep Well Assays in the current examples were performed as originally described to Lv et al (Bioprocess Biosyst Eng 20 (2016) 39:1737-1747) and optimized for the purposes of the current invention. More specifically, the strains disclosed in the present example were screened in 96 deep well plates using a 4-day protocol. During the first 24 hours, precultures were grown to high densities (OD600 up to 5) and subsequently transferred to a medium that allowed induction of gene expression and product formation.

More specifically, during day 1, fresh precultures were prepared using a basal minimal medium (BMM) (pH 7.0) supplemented with magnesium sulphate (0.12 g/L), thiamine (0.004 g/L) and glucose (5.5 g/L). Basal Minimal medium had the following composition: NaOH (1 g/L), KOH (2.5 g/L), KHzPO4 (7 g/L), NH&HzPO4 (7 g/L), Citric acid (0.5 g/l), trace mineral solution (5 mL/L). The trace mineral stock solution contained; ZnSO~*7H~O 0.82 g/L, Citric acid 20 g/L, Mn$04*H&O 0.98 g/L, FeSO4*7H&O 3.925 g/L, CuSO4*5H~O 0.2 g/L. The pH of the Basal Minimal Medium was adjusted to 7.0 with 5 N NaOH and autoclaved. The precultures were incubated for 24 hours at 34° C. and 1000 rpm shaking and then further transferred to 0.75 mL of a new BMM (pH 7.5) to start the main culture. The new BMM was supplemented with magnesium sulphate (0.12 g/L), thiamine (0.02 g/L), a bolus of glucose solution (0.1-0.15 g/L) and a bolus of lactose solution (5-20 g/L) Moreover, a 20% stock solution of sucrose (40-45 g/L) or maltodextrin (19-20 g/L) was provided as carbon source, accompanied by the addition of a specific hydrolytic enzyme, sucrose hydrolase or glucoamylase, respectively, so that glucose was released at a rate suitable for carbon-limited growth and similar to that of a typical fed-batch fermentation process. The main cultures were incubated for 72 hours at 28° C. and 1000 rpm shaking. For the analysis of total broth, the 96 well plates were boiled at 100° C., subsequently centrifuged, and finally the supernatants were analysed by HPLC.

### Fermentation

The E. coli strains were cultivated in 250 mL fermenters (Ambr250 HT Bioreactor system, Sartorius) starting with 100 mL of mineral culture medium consisting of 30 g/L glucose and a mineral medium comprised of NH₄H₂PO₄, KH₂PO₄, MgSO₄×7H₂O, KOH, NaOH, citric acid, trace element solution, antifoam and thiamine. The dissolved oxygen level was kept at 20% by a cascade of first agitation and then airflow starting at 700 rpm (up to max 4500 rpm) and 1 VVM (up to max 3 VVM). The pH was kept at 6.8 by titration with 8.5% NH₄OH solution. The cultivations were started with 2% (v/v) inoculums from pre-cultures comprised of 10 g/L glucose, (NH₄)₂HPO₄, KH₂PO₄, MgSO4×7H₂O, KOH, NaOH, citric acid, trace element solution, antifoam and thiamine. After depletion of the glucose contained in the basal minimal medium, a feed solution containing glucose, MgSO₄×7H₂O, H₃PO₄ and trace mineral solution was continuously added to the fermenter at a rate that maintained carbon-limiting conditions. The temperature was initially at 33° C. but was dropped to 30° C. with a 3-hour linear ramp initiated 12 hours after the start of the feed. Lactose was added as bolus additions of 25% lactose monohydrate solution 6 hours after feed start and then every 19 hours to keep lactose from becoming a rate limiting factor. The growth, metabolic activity and metabolic state of the cells was followed by on-line measurements of agitation, dissolved oxygen tension, reflectance, NH₄OH base addition, O₂ uptake rate and CO₂ evolution rate. Throughout the fermentations, samples were taken to determine the concentration of HMO products, lactose and other minor by-products using HPLC.

### Example 1—In Vivo LNDFH-II Synthesis

Genetically modified cells expressing individual α-1,3/4-fucosyltransferase enzymes were screened for their ability to produce the fucosylated HMO LNDFH-II and mixtures thereof.

Four enzymes (Table 6) were compiled for testing their ability to synthesize LNDFH-II when introduced into a genetically modified cell that produce LNT and GDP-Fucose. The enzymes were compared to the known α-1,3-fucosyltransferase enzyme, FutA.

Genetically modified strains expressing the five individual α-1,3/4-fucosyltransferases (Table 4) were generated as described in the “Method” section. The cells were screened in the deep well assay setup as described in the “Method” section.

Table 5 lists the genotype of the strains capable of producing LNDFH-II. The molar content of individual HMOs produced by the strains was measured by HPLC.

The results of the LNDFH-II producing cells are shown in table 6 as the fraction of the total molar HMO content (in percentage, %) produced by each strain.

TABLE 6
Content of individual HMO's as % of total
HMO molar (mM) content produced by each strain.

| | FT | | | | | | | |
|---|---|---|---|---|---|---|---|---|
| Enzyme | Copy nr | LNT2 | 3FL | LNT | LNFP-II | LNFP-V | LNDFH-II | pLNH2 |
| Bgall1-1 | 1 | 0 | 21 | 9 | 0 | 54 | 16 | 0 |
| Bgall1-2 | 2 | 0 | 38 | 3 | 0 | 35 | 24 | 0 |
| Bgall1-3 | >100 | 0 | 58 | 2 | 0 | 23 | 16 | 0 |
| Murba1-1 | 1 | 0 | 11 | 23 | 0 | 66 | 1 | 0 |
| Murba1-2 | 2 | 0 | 22 | 10 | 0 | 64 | 4 | 0 |
| Murba1-3 | >100 | 0 | 50 | 3 | 0 | 40 | 7 | 0 |
| Bbac1 | 1 | 0 | 23 | 3 | 0 | 67 | 7 | 2 |
| Para1-1 | 1 | 38 | 1 | 51 | 4 | 4 | 2 | 0 |
| Para1-2 | 2 | 45 | 0 | 34 | 5 | 5 | 11 | 0 |
| FutA-1 | 1 | 0 | 0 | 20 | 0 | 75 | 5 | 0 |
| FutA-2 | 2 | 0 | 0 | 8 | 0 | 79 | 13 | 0 |
| FutA-3 | >100 | 0 | 17 | 3 | 0 | 42 | 39 | 0 |

No additional HMOs beyond the ones indicated in Table 6 was identified in the deep well assay.

From the data presented in Table 6 it can be seen that a single copy of the two enzymes Bgall1 and Bbac1, can transfer a fucosyl unit onto the Glc and GlcNAc moieties of LNT in an α-1,3 linkage at the Glc moiety and in an α-1,4 linkage at the GlcNAc moiety to form LNDFH-II at a level above 7% of the total HMO. FutA which is known as an α-1,3-fucosyltransferase in the prior art (see for example Dumon et al., 2004 (Biotechnol. Prog. 2004, 20, 412-419) was surprisingly found to be able to fucosylate the GlcNAc moiety of LNT in an α-1,4 linkage and produced 5% LNDFH-II of the total HMO, respectively, when expressed from a single genomic copy under control of the PglpF promoter.

From table 6 it can be seen that increasing the copy number of Bgall1 to two genomic copies increased the relative amount of LNDFH-II produced from 16% to 24% of the total HMO content, combined with a decrease in the relative LNFP-V amount produced, from 54% to 37%, a slight decrease in the relative LNT amount produced, from 9% to 3%, and an increase in the amount of 3FL produced from 21% to 38% respectively. A further increase in the expression level of Bgall1 by introduction of a high copy plasmid encoding Bgall1, decreased the relative amount of LNDFH-II produced from 24% to 16% of the total HMO content, combined with a further decrease in the relative LNFP-V amount produced, from 37% to 23%, a slight decrease in the relative LNT amount produced, from 3% to 2%, and a further increase in the amount of 3FL produced from 38% to 54%, respectively.

Increasing the copy number of Murba1 to two genomic copies increased the relative amount of LNDFH-II produced from 1% to 4% of the total HMO content, combined with a decrease in the relative LNFP-V amount produced, from 66% to 64%, a slight decrease in the relative LNT amount produced, from 23% to 10%, and an increase in the amount of 3FL produced from 11% to 22% respectively. A further increase in the expression level of Murba1 by introduction of a high copy plasmid encoding Murba1, further increased the relative amount of LNDFH-II produced from 4% to 7% of the total HMO content, combined with a further decrease in the relative LNFP-V amount produced, from 64% to 40%, a decrease in the relative LNT amount produced, from 10% to 3%, and a further increase in the amount of 3FL produced from 22% to 50%, respectively.

Increasing the copy number of Para1 to two genomic copies increased the relative amount of LNDFH-II produced from 2% to 11% of the total HMO content, combined with a minor increase in the relative LNFP-II amount produced, from 4% to 5%, a minor increase in the relative LNFP-V amount produced, from 4% to 5%, a decrease in the relative LNT amount produced, from 51% to 34%, a decrease in the amount of 3FL produced from 1% to 0% and an increase in LNT2 produced from 38% to 45%, respectively.

Increasing the copy number of FutA to two genomic copies increased the relative amount of LNDFH-II produced from 5% to 13% of the total HMO content, combined with an increase in the relative LNFP-V amount produced, from 75% to 79% and a slight decrease in the relative LNT amount produced, from 20% to 8% respectively. A further increase in the expression level of FutA by introduction of a high copy plasmid encoding FutA, further increased the relative amount of LNDFH-II produced from 13% to 39% of the total HMO content, combined with a decrease in the relative LNFP-V amount produced, from 79% to 42% and a slight decrease in the relative LNT amount produced, from 8% to 3% respectively.

Accordingly, the copy number variation of Bgall1, Para1 and FutA may be used to tailor specific HMOs mixtures, in this case a mixture comprising of LNDFH-II and LNT2, 3FL, LNT, LNFP-II and/or LNFP-V, in different ratios, depending on the need for the specific product.

Bbac1 was found to produce mixture of HMOs consisting of 97% fucosylated HMOs, where only 3% LNT was produced, and the remaining 97% of the produced HMOs were a mixture of 3FL, LNDFH-II and LNFP-V. Bbac1 would therefore be a good choice for a fucosylated HMO mixture with low levels on non-fucosylated HMOs such as LNT-II and LNT.

Strains containing high copy number plasmids with either Bgall1, Murba1 or FutA was also shown to be able to produce mixtures of fucosylated HMOs, wherein the content of fucosylated HMOs exceeded 90% of the total HMOs produced, and even as high as 98% of the total HMOs produced by the Bgall1-3 strain was fucosylated HMOs.

### Example 2—Fermentation of Bgall1 or FutA Strains for LNDFH-II Production

To confirm the HMO profile observed in the deep well assays, and especially the content of LNDFH-II in the total HMO produced, the Bgall1-2, Murba1-2, FutA-1 and FutA-2 strains of example 1, containing either two genomic copies of Bgall1, or one or two genomic copies of FutA were fermented as described in the “Method” section above. The results are shown in table 7.

TABLE 7
Content of individual HMO's as % of
total HMO content produced by the strain

| | FT | | | | | total HMO |
|---|---|---|---|---|---|---|
| Strain ref. | Copy nr | 3FL | LNT | LNFP-V | LNDFH-II | (%) |
| FutA-1 | 1 | 5 | 6 | 77 | 13 | 100 |
| FutA-2 | 2 | 6 | 4 | 67 | 23 | 100 |
| Bgall1-2 | 2 | 55 | 0 | 19 | 26 | 100 |
| Murba1-2 | 2 | 34 | 4 | 55 | 7 | 100 |

From the data presented in table 7, it can be seen that the fraction of LNDFH-II for the FutA-1, and FutA-2 strains were increased in the fermentation compared to the deep well assay. For the Bgall1-2 strain the LNDFH-II content increased slightly in the fermentation compared to the deep well assay the results presented in example 1. The Murba1-2 strain almost doubled the LNDFH-II levels produced in the fermentation as compared to the deep well assay presented in example 1, although the levels were lower than for Bgall1 and FutA it could be a strain of interest if a mixture with high levels of LNFP-V and 3FL is desired in combination with some LNDFH-II.

### Example 3—Comparative Example of Bgall1, FutA and FucTIII Strains for LNDFH-II Production

Considering the disclosure in Baumgärtner et al 2015 (Bioorganic & Medicinal Chemistry 23:6799-6806) a comparative example using the DW assay described in the Methods section was performed.

Table 5 lists the genotype of the strains used in the current example. The molar content of individual HMOs produced by the strains was measured by HPLC.

The results of the LNDFH-II producing cells are shown in table 8 as the fraction of the total molar HMO content (in percentage, %) produced by each strain.

TABLE 8
Content of individual HMO's as % of total
HMO molar (mM) content produced by each strain.

| | FT | | | | | | | |
|---|---|---|---|---|---|---|---|---|
| Enzyme | Copy nr | 3FL | LNT-II | LNT | LNFP-II | LNFP-V | LNDFH-II | pLNH2 |
| Bgall1-1 | 1 | 17 | 19 | 7 | 0 | 43 | 14 | 0 |
| FutA-1 | 1 | 0 | 27 | 21 | 0 | 50 | 3 | 0 |
| FucTIII | 1 | 0 | 14 | 78 | 1 | 5 | 0 | 2 |

From the results in table 8 it can be seen that Bgall1 under the current conditions by far is the best producer of LNDFH-II. Surprisingly, FucTIII despite the report in Baumgartner does not produce any LNDFH-II at all.

1. A genetically engineered cell capable of producing LNDFH-II, comprising a recombinant nucleic acid sequence encoding a fucosyltransferase, wherein the fucosyltransferase is selected from the group consisting of,
a. Bgall1 comprising an amino acid sequence according to SEQ ID NO: 1, or a functional homologue thereof with an amino acid sequence that is at least 80% identical to SEQ ID NO: 1,
b. Murba1 comprising an amino acid sequence according to SEQ ID NO: 2, or a functional homologue thereof with an amino acid sequence that is at least 80% identical to SEQ ID NO: 2,
c. Bbac1 comprising an amino acid sequence according to SEQ ID NO: 3, or a functional homologue thereof with an amino acid sequence that is at least 80% identical to SEQ ID NO: 3, and
d. Para1 comprising an amino acid sequence according to SEQ ID NO: 4, or a functional homologue thereof with an amino acid sequence that is at least 80% identical to SEQ ID NO: 4, and
e. FutA comprising an amino acid sequence according to SEQ ID NO: 5, or a functional homologue thereof with an amino acid sequence that is at least 80% identical to SEQ ID NO: 5.

2. The genetically engineered cell according to claim 1, wherein the cell further comprises one or more recombinant nucleic acid sequence(s) encoding a β-1,3-galactosyltransferase.

3. The genetically engineered cell according to 1, wherein the cell further comprises a substrate importer selected from a lactose importer, a lacto-N-triose-II (LNT-II) importer, or an LNT importer.

4. The genetically engineered cell according to claim 1, wherein the cell, in fermentative conditions, produces one or more HMOs selected from the group consisting of 3FL, LNT-II, LNT and LNFP-V.

5. The genetically engineered cell according to claim 1, wherein the cell comprises two or more genomic copies or a plasmid-borne copy of the recombinant nucleic acid sequence encoding the fucosyltransferase.

6. The genetically engineered cell according to claim 1, wherein the engineered cell is selected from the group consisting of Escherichia Coli, Bacillus subtilis, Lactobacillus lactis, Corynebacterium glutamicum, Yarrowia lipolytica, Pichia pastoris, and Saccharomyces cerevisiae.

7. A method for producing one or more fucosylated HMOs comprising the following steps of:
providing the genetically modified cell according to claim 1
b. culturing the cell according to (a) in a suitable cell culture medium to produce said one or more fucosylated HMOs, wherein at least one of the fucosylated HMOs is LNDFH-II, and
c. optionally, purifying said one or more fucosylated HMOs.

8. The method according to claim 7, wherein at least 5% of the molar content of the total HMOs produced by the genetically engineered cell cultivated in said method is LNDFH-II.

9. The method according to claim 7, wherein at least 65% of the molar content of the total HMOs produced by the method are fucosylated HMOs selected from the group consisting of 3FL, LNFP-V and LNDFH-II.

10. (canceled)

11. The method according to claim 7, wherein the genetically engineered cell is cultivated in the presence of an acceptor substrate selected from the group consisting of lactose, LNT-II and LNT.

12. (canceled)

13. The genetically engineered cell according to claim 4, wherein the cell, in fermentative conditions, produces a mixture of HMOs consisting essentially of
a. LNDFH-II, LNFP-V and LNT,
b. LNDFH-II, LNFP-V and 3FL,
c. LNDFH-II, LNFP-V, LNT and 3FL, or
d. LNDFH-II, LNT-II, LNFP-V, LNFP-II, and LNT.

14. A composition of HMOs consisting essentially of 1-40 molar % of LNDFH-II, 2-85 molar % LNFP-V, 0-10 molar % LNFP-II, 0-65 molar % 3FL, 0-55 molar % LNT and 0-50 molar % LNT-II, in total adding up to 100% molar content.

15. The composition according to claim 14, wherein the mixture is selected from the group consisting of:
a. a mixture consisting essentially of 15-40 molar % of LNDFH-II and 20-60 molar % LNFP-V, 0-15 molar % LNT and 15-60% 3FL, in total adding up to 100% molar content;
b. a mixture consisting essentially of 15-40 molar % of LNDFH-II and 15-40 molar % LNFP-V, 0-5 molar % LNT and 25-60% 3FL, in total adding up to 100% molar content;
c. a mixture consisting essentially of 1-15 molar % of LNDFH-II and 35-70 molar % LNFP-V, 0-25 molar % LNT and 5-55% 3FL, in total adding up to 100% molar content;
d. a mixture consisting essentially of 2-15 molar % of LNDFH-II and 60-75 molar % LNFP-V, 0-10 molar % LNT and 15-30% 3FL, in total adding up to 100% molar content; and
e. a mixture consisting essentially of 2-15 molar % of LNDFH-II and 0-10 molar % LNFP-V, 0-10 molar % LNFP-II, 30-50 molar % LNT-II, 30-60 molar % LNT and 0-5 molar % 3FL, in total adding up to 100% molar content, and
f. a mixture consisting essentially of 2-40 molar % of LNDFH-II and 35-85 molar % LNFP-V, 0-25 molar % LNT and 0-20% 3FL, in total adding up to 100% molar content.

16. (canceled)

17. The genetically engineered cell according to claim 1, wherein the cell further comprises one or more recombinant nucleic acid sequence(s) encoding a β-1,3-N-acetylglucosaminyltransferase.

18. The genetically engineered cell according to claim 1, wherein the cell further comprises recombinant nucleic acid sequences encoding a β-1,3-N-acetylglucosaminyltransferase and a β-1,3-galactosyltransferase.

19. The genetically engineered cell according to claim 18, wherein the cell further comprises a lactose importer.

20. The genetically engineered cell according to claim 19, wherein the cell, in fermentative conditions, produces LNDFH-II and LNFP-V.

21. The genetically engineered cell according to claim 20, wherein the cell, in fermentative conditions, further produces at least one additional HMO selected from the group consisting of LNT, 3FL, LNT-II, and LNFP-II.

22. The genetically engineered cell according to claim 20, wherein the cell, in fermentative conditions, further produces at least one of the HMOs 3FL or LNT.


SEQUENCE LISTING (40 sequences)

SEQ ID NO: 1 - protein, 321 aa, Bacteroides gallinaceum
MKSLKIKFVDFWPGFNPNDNFITNALKGYEIVITDTPDYLFFSIFGYSHLKYNCVKIMFVGENIVPDFNLCDYAMGFDFLNFGDRYMRLPLFLICDNFKELSVTKDFSPKRMLNRKFCSIVVSNAQVSNPIRERFFRLLSEYKQVDSGGRLWNNVGGPVADKQKFISGYKFNIAFENSAVLGYTTEKIMDAMTANTLPIYWGNPWVGRDFNKRSFVNVNSFASLEKAVEYIVDLDTNDERYLEMMQEPWVNDVSIFDWEDKLCAFLAHIVEKPFAEAQYLVDDGMQKLYKQNMKTLAFVNEKLKVPRLISAYKKLSNGKYK

SEQ ID NO: 2 - protein, 320 aa, Muribaculaceae bacterium
MRDVTVAFVDFWPAFDDKNNFILDVLRQKYNVKTVAASDCPDILFYSYFGIENLRHTEPVRVYFTGENDVPDFNLCDYAISFHHLSFANRHLRLPLYVIYSCFNDLRNGRILQCEDAENRDFCSMVVSNVKNSDMARLDFFNLLSGYKQVASGGRFANNVGGCVTDKLSFISGYKFNIAFENSRVEGYVTEKIIEAFSSGTLPIYWGDPLVANELNDKAFIDVSKFDSLDAAVKFVAALDNNREQYLEILNQPPLLNNPFLNWEEMLLDFLTPIVNELKKYRVDTGAIGTAYRHKMSREQIYNIVTLKKPFELFKHLFHS

SEQ ID NO: 3 - protein, 323 aa, Bacteroidaceae bacterium
MNNKIKINFVDFWGDFNKCDNFITRALSKKYEVAISDNPDYLFFGTFGYRHLNYNCVKIMFIGENIAPDFNLCDYALGFDFIQFGDRYMRLPLYCTYESFASLAQTRLPSDEALIDRKFCSMVVSNNRHASPHRERFFKLLSEYKQVDSGGRLWNNVGGPVANKLEFVSQYKFNIAFENSAVHGYTTEKIMEPMAVNTLPLYWGNPLVCKDFNAASFVNVNDFASMEDAVQHIVDLDNDDSKYLQMIKAPKVLDENIINWEERLLDFLSNIIEKPIEEARYLAPFGVQKLYREELLAYDILSRRLKVKKIVAAYDKLRKKIRG

SEQ ID NO: 4 - protein, 314 aa, Parabacteroides sp.
MKKIKINFVDFWDGFNCFDNYFVNILTCYYDVEIDAKPDYLFYSVFGDNHLEYKNCIKIYYTGENDFPDFNFCDYAIGFQYFTFEDRSFRYPEYLLYEGYNKLMDKEKTIDKLLSNRKFCNFVYSNSKYAIPLREKFFHQLSKYKKVDSGGRLLNNVGGPVQNKLDFIKDYKFTIAFENSSLSGYTTEKLMEPMVVNSLPIYWGNPNVHLDFNVASIINLQSFSSFEEAIEEIVRLDNDDDAYLQKLSQPWLTQDQAEKNWEKELALFLCNIFDQPIEKARRRTDFGFGVMKKFREARFSRFLSYSIFRRLLNF

SEQ ID NO: 5 - protein, 425 aa, Helicobacter pylori
MFQPLLDAFIESASIEKMASKSPPPPLKIAVANWWGDEEIKEFKKSVLYFILSQRYAITLHQNPNEFSDLVFSNPLGAARKILSYQNTKRVFYTGENESPNFNLFDYAIGFDELDFNDRYLRMPLYYAHLHYKAELVNDTTAPYKLKGNSLYALKKPSHHFKENHPNLCAVVNDESDLLKRGFASFVASNANAPMRNAFYDALNSIEPVTGGGSVRNTLGCKVGNKSEFLSQYKFNLCFENSQGYGYVTEKILDAYFSHTIPIYWGSPSVAKDFNPKSFVNVHDFNNFDEAIDYIKYLHTHPNAYLDMLYENPLNTLDGKAYFYQDLSFKKILDFFKTILENDTIYHKFSTSFMWEYDLHKPLVSIDDLRVNYDDLRVNYDRLLQNASPLLELSQNTTFKIYRKAYQKSLPLLRAVRKLVKKLGL

SEQ ID NO: 6 - DNA, 966 bp, Synthetic construct
  source 1..966: mol_type = other DNA
atgaaaagcctgaagatcaagttcgtggatttttggcctggttttaatccgaacgataactttattaccaatgcgctgaaaggctatgagattgtgattaccgatacaccggattacctgtttttcagcatttttggttatagccacctgaagtacaactgcgtgaaaattatgtttgtgggcgaaaatatcgtgccggattttaatctgtgtgattatgccatgggcttcgactttctgaattttggtgatcgttatatgcgtctgccgctgtttctgatttgcgataatttcaaagaactgagcgtgaccaaagattttagcccgaaacgtatgctgaatcgcaaattttgtagcattgttgttagcaatgcccaggttagcaatccgattcgtgaacgtttctttcgtctgctgagcgaatataaacaggttgatagcggtggtcgtctgtggaataatgttggtggtccggttgcagataaacagaaatttatctccggctataaattcaacattgcctttgaaaatagcgcagtgctgggttataccaccgaaaagattatggatgcaatgaccgcaaataccctgccgatttattggggtaatccgtgggttggtcgtgatttcaataaacgtagctttgtgaacgtgaatagctttgcaagcctggaaaaagccgttgaatatattgttgatctggataccaacgatgagcgctatctggaaatgatgcaagaaccgtgggttaatgatgtgtccatttttgactgggaagataaactgtgtgcatttctggcacatattgtcgaaaaaccgtttgcagaagcacagtatctggttgatgatggtatgcagaaactgtacaagcagaatatgaaaaccctggcctttgtgaatgagaaactgaaagttccgcgtctgattagcgcatataagaaactgagcaacggcaagtacaaataa

SEQ ID NO: 7 - DNA, 963 bp, Synthetic construct
  source 1..963: mol_type = other DNA
atgcgtgatgttaccgttgcctttgttgatttttggcctgcctttgatgacaaaaacaactttattctggatgtgctgcgccagaaatataacgttaaaaccgttgcagcaagcgattgtccggatattctgttttatagctatttcggcattgaaaacctgcgtcataccgaaccggtgcgtgtgtattttacaggtgaaaatgatgtgccggatttcaacctgtgtgattatgcaattagctttcaccatctgagctttgcaaatcgtcatctgcgtctgccgctgtatgtgatttatagctgttttaatgatctgcgcaatggtcgtattctgcagtgtgaagatgcagaaaatcgtgatttttgcagcatggttgtgagcaatgtgaaaaatagcgatatggcacgtctggatttctttaatctgctgagcggttataaacaggttgcaagcggtggtcgttttgcaaataatgttggtggttgtgttaccgacaaactgagctttattagcggctacaaattcaacattgcctttgaaaattcacgcgtggaaggttatgtgaccgagaaaattatcgaagcatttagcagcggcaccctgccgatttattggggtgatccgctggttgcaaatgaactgaatgataaagccttcatcgacgtgagcaaatttgatagcctggatgcagcagttaaatttgttgcagcactggataataaccgcgaacagtatctggaaattctgaatcagcctccgctgctgaataatccgtttctgaattgggaagaaatgctgctggattttctgaccccgattgttaacgaactgaaaaagtatcgtgttgataccggtgcaattggtacagcatatcgtcataaaatgagccgtgagcagatttataacattgtgaccctgaaaaagccgttcgaactgttcaaacacctgtttcacagctaa

SEQ ID NO: 8 - DNA, 972 bp, Synthetic construct
  source 1..972: mol_type = other DNA
atgaacaacaagatcaagatcaacttcgtggatttctggggtgatttcaacaaatgcgataactttattacccgtgcgctgagcaaaaagtatgaagttgcaattagcgataacccggattacctgtttttcggcacctttggttatcgtcatctgaattataactgcgtgaaaatcatgttcatcggcgaaaatattgcaccggattttaacctgtgtgattatgcactgggctttgatttcattcagtttggtgatcgttatatgcgtctgccgctgtattgtacctatgaaagctttgcaagcctggcacagacccgtctgccgagtgatgaagcactgattgatcgtaaattttgcagcatggtggtgagcaataatcgtcatgcaagtccgcatcgtgaacgtttctttaaactgctgagcgaatataaacaggttgatagcggtggtcgtctgtggaataatgttggtggtccggttgcaaataaactggaatttgttagccagtacaaattcaacattgcctttgaaaatagcgcagtgcatggttataccaccgagaagattatggaaccgatggcagttaataccctgccgttatattggggtaatccgctggtttgcaaagactttaatgcagccagctttgtgaacgtgaatgattttgcaagtatggaagatgccgttcagcatattgttgatctggataacgatgacagcaaatacctgcagatgattaaagcaccgaaagtgctggatgaaaacatcattaattgggaagaacgcctgctggattttctgagcaatattatcgaaaaaccgatcgaggaagcacgttatctggcaccgtttggtgttcagaaactgtatcgtgaagaactgctggcatatgatattctgagccgtcgtctgaaagtgaaaaagattgttgcagcctatgataaactgcgcaaaaagattcgtggctaa

SEQ ID NO: 9 - DNA, 945 bp, Synthetic construct
  source 1..945: mol_type = other DNA
atgaagaagatcaagatcaacttcgtggatttttgggatggctttaactgcttcgataactactttgtgaatatcctgacctgctattacgatgtggaaattgatgcaaaaccggactacctgttttatagcgtgtttggtgataaccacctggaatacaaaaactgcatcaagatctattacaccggtgaaaacgatttcccggattttaacttttgcgattatgccatcggcttccagtattttacctttgaagatcgtagctttcgctatccggaatatctgctgtatgaaggctataacaaactgatggacaaagagaaaaccatcgataaactgctgagcaaccgcaaattttgcaactttgtttacagcaacagcaaatatgcaattccgctgcgcgaaaaattctttcatcagctgagcaaatacaagaaagttgatagcggtggtcgtctgctgaataatgttggtggtccggttcagaataaactggatttcatcaaagactataaattcaccattgccttcgaaaatagcagcctgagcggttataccaccgaaaagctgatggaaccgatggttgttaatagcctgccgatttattggggtaatccgaatgttcacctggattttaatgttgccagcattattaacctgcagagctttagcagctttgaagaagccattgaagaaattgtgcgcctggataatgatgatgatgcatatctgcagaaactgagccagccgtggctgacccaggatcaggccgagaaaaattgggaaaaagaactggcactgtttctgtgcaacatttttgatcagccgattgaaaaagcacgtcgtcgtaccgattttggttttggtgttatgaaaaagtttcgcgaagcacgttttagccgttttctgagctatagcatttttcgtcgcctgctgaacttttaa

SEQ ID NO: 10 - DNA, 1278 bp, Synthetic construct
  source 1..1278: mol_type = other DNA
atgttccaaccgctgctggacgcgttcatcgaatctgcctctattgaaaaaatggcctcgaaatcgccgccgccgccgctgaaaatcgcagtggctaattggtggggtgatgaagaaatcaaagaatttaaaaaatctgtgctgtatttcattctgtctcagcgttacgcaatcaccctgcatcaaaacccgaatgaatttagtgacctggtcttctccaacccgctgggtgcagcacgtaaaattctgagctatcagaataccaaacgcgtgttttacacgggtgaaaacgaatctccgaactttaacctgtttgattatgccatcggctttgatgaactggacttcaatgatcgttatctgcgcatgccgctgtattacgcacatctgcactataaagctgaactggttaatgataccacggcgccgtataaactgaagggtaacagtctgtacgccctgaaaaaaccgtcccatcactttaaagaaaaccacccgaatctgtgcgcggtggttaacgacgaaagcgatctgctgaaacgtggctttgcatcattcgttgcttcgaacgcgaatgccccgatgcgcaacgcgttttatgatgccctgaacagcattgaaccggttaccggcggtggctcggtccgtaatacgctgggttgcaaagtgggcaacaaaagcgaatttctgtctcagtacaaattcaacctgtgtttcgaaaatagtcaaggttatggctacgttaccgaaaaaatcctggatgcgtatttctcccatacgattccgatctactggggtagcccgtctgtcgccaaagattttaacccgaaatctttcgtcaatgtgcacgacttcaacaacttcgacgaagcaatcgattacatcaaatacctgcatacccacccgaatgcttatctggatatgctgtacgaaaacccgctgaatacgctggacggcaaagcctatttttaccaggatctgagtttcaagaaaattctggatttctttaaaaccattctggaaaacgatacgatctaccataaattcagtaccagctttatgtgggaatacgacctgcacaaaccgctggttagcatcgatgacctgcgtgttaactatgatgacctgcgtgtcaattacgatcgcctgctgcagaacgcatcaccgctgctggaactgtcgcaaaataccacgtttaaaatctatcgcaaagcgtatcaaaaatcactgccgctgctgcgtgctgtccgtaaactggtcaaaaaactgggtctgtaa

SEQ ID NO: 11 - DNA, 6706 bp, Synthetic construct
  source 1..6706: mol_type = other DNA
atgtcaaaagtcgctctcatcaccggtgtaaccggacaagacggttcttacctggcagagtttctgctggaaaaaggttacgaggtgcatggtattaagcgtcgcgcatcgtcattcaacaccgagcgcgtggatcacatttatcaggatccgcacacctgcaacccgaaattccatctgcattatggcgacctgagtgatacctctaacctgacgcgcattttgcgtgaagtacagccggatgaagtgtacaacctgggcgcaatgagccacgttgcggtctcttttgagtcaccagaatataccgctgacgtcgacgcgatgggtacgctgcgcctgctggaggcgatccgcttcctcggtctggaaaagaaaactcgtttctatcaggcttccacctctgaactgtatggtctggtgcaggaaattccgcagaaagagaccacgccgttctacccgcgatctccgtatgcggtcgccaaactgtacgcctactggatcaccgttaactaccgtgaatcctacggcatgtacgcctgtaacggaattctcttcaaccatgaatccccgcgccgcggcgaaaccttcgttacccgcaaaatcacccgcgcaatcgccaacatcgcccaggggctggagtcgtgcctgtacctcggcaatatggattccctgcgtgactggggccacgccaaagactacgtaaaaatgcagtggatgatgctgcagcaggaacagccggaagatttcgttatcgcgaccggcgttcagtactccgtgcgtcagttcgtggaaatggcggcagcacagctgggcatcaaactgcgctttgaaggcacgggcgttgaagagaagggcattgtggtttccgtcaccgggcatgacgcgccgggcgttaaaccgggtgatgtgattatcgctgttgacccgcgttacttccgtccggctgaagttgaaacgctgctcggcgacccgaccaaagcgcacgaaaaactgggctggaaaccggaaatcaccctcagagagatggtgtctgaaatggtggctaatgacctcgaagcggcgaaaaaacactctctgctgaaatctcacggctacgacgtggcgatcgcgctggagtcataagcatgagtaaacaacgagtttttattgctggtcatcgcgggatggtcggttccgccatcaggcggcagctcgaacagcgcggtgatgtggaactggtattacgcacccgcgacgagctgaacctgctggacagccgcgccgtgcatgatttctttgccagcgaacgtattgaccaggtctatctggcggcggcgaaagtgggcggcattgttgccaacaacacctatccggcggatttcatctaccagaacatgatgattgagagcaacatcattcacgccgcgcatcagaacgacgtgaacaaactgctgtttctcggatcgtcctgcatctacccgaaactggcaaaacagccgatggcagaaagcgagttgttgcagggcacgctggagccgactaacgagccttatgctattgccaaaatcgccgggatcaaactgtgcgaatcatacaaccgccagtacggacgcgattaccgctcagtcatgccgaccaacctgtacgggccacacgacaacttccacccgagtaattcgcatgtgatcccagcattgctgcgtcgcttccacgaggcgacggcacagaatgcgccggacgtggtggtatggggcagcggtacaccgatgcgcgaatttctgcacgtcgatgatatggcggcggcgagcattcatgtcatggagctggcgcatgaagtctggctggagaacacccagccgatgttgtcgcacattaacgtcggcacgggcgttgactgcactatccgcgagctggcgcaaaccatcgccaaagtggtgggttacaaaggccgggtggtttttgatgccagcaaaccggatggcacgccgcgcaaactgctggatgtgacgcgcctgcatcagcttggctggtatcacgaaatctcactggaagcggggcttgccagcacttaccagtggttccttgagaatcaagaccgctttcgggggtaatgatgtttttacgtcaggaagactttgccacggtagtgcgctccactccgcttgtctctctcgactttattgtcgagaacagtcgcggcgagtttctgcttggcaaaagaaccaaccgcccggcgcagggttactggtttgtgccgggagggcgcgtgcagaaagacgaaacgctggaagccgcatttgagcggctgacgatggcggaactggggctgcgtttgccgataacagcaggccagttttacggtgtctggcagcacttttatgacgataacttctctggcacggatttcaccactcactatgtggtgctcggttttcgcttcagagtatcggaagaagagctgttactgccggatgagcagcatgacgattaccgctggctgacgtcggacgcgctgctcgccagtgataatgttcatgctaacagccgcgcctattttctcgctgagaagcgtaccggagtacccggattatgaaaatactggtctacggcattaactactcgccggagttaaccggcatcggcaaatacaccggcgagatggtggaatggctggcggcacaaggtcatgaggtgcgggtcattaccgcaccgccttactacccgcaatggcaggtgggcgagaactattccgcctggcgctacaaacgagaagagggggccgccacggtgtggcgctgcccgctgtatgtgccaaaacagccgagcaccctgaaacgcctgttgcatctgggcagttttgccgtcagcagtttctttccgctgatggcgcaacgtcgctggaagccggatcgcattattggcgtggtgccaacgctgttttgcgcgccgggaatgcgcctgctggcgaaactctctggtgcgcgtaccgtgctgcatattcaggattacgaagtggacgccatgctggggctgggccttgccggaaaaggcaaaggcggcaaagtggcacagctggcaacggcgttcgaacgtagcggactgcataacgtcgataacgtctccacgatttcgcgttcgatgatgaataaagccatcgaaaaaggcgtggcggcggaaaacgtcatcttcttccccaactggtcggaaattgcccgttttcagcatgttgcagatgccgatgttgatgcccttcgtaaccagcttgacctgccggataacaaaaaaatcattctttactccggcaatattggtgaaaagcaggggctggaaaacgttattgaagctgccgatcgtctgcgcgatgaaccgctgatttttgccattgtcgggcagggcggcggcaaagcgcggctggaaaaaatggcgcagcagcgtggactgcgcaacatgcaatttttcccgctgcaatcgtatgacgctttacccgcactgctgaagatgggcgattgccatctggtggtgcaaaaacgcggcgcggcagatgccgtattgccgtcgaaactgaccaatattctggcagtaggcggtaacgcggtgattactgctgaagcctacacagaactggggcagctttgcgaaacctttccgggcattgcggtttgcgttgaaccggaatcggtcgaggcgctggtggcggggatccgtcaggcgctcctgctgcccaaacacaacacggtggcacgtgaatatgccgaacgcacgctcgataaagagaacgtgttacgtcaatttataaatgatattcggggataattatggcgcagtcgaaactctatccagttgtgatggcaggtggctccggtagccgcttatggccgctttcccgcgtactttatcccaagcagtttttatgcctgaaaggcgatctcaccatgctgcaaaccaccatctgccgcctgaacggcgtggagtgcgaaagcccggtggtgatttgcaatgagcagcaccgctttattgtcgcggaacagctgcgtcaactgaacaaacttaccgagaacattattctcgaaccggcagggcgaaacacggcacctgccattgcgctggcggcgctggcggcaaaacgtcatagcccggagagcgacccgttaatgctggtattggcggcggatcatgtgattgccgatgaagacgcgttccgtgccgccgtgcgtaatgccatgccatatgccgaagcgggcaagctggtgaccttcggcattgtgccggatctaccagaaaccggttatggctatattcgtcgcggtgaagtgtctgcgggtgagcaggatatggtggcctttgaagtggcgcagtttgtcgaaaaaccgaatctggaaaccgctcaggcctatgtggcaagcggcgaatattactggaacagcggtatgttcctgttccgcgccggacgctatctcgaagaactgaaaaaatatcgcccggatatcctcgatgcctgtgaaaaagcgatgagcgccgtcgatccggatctcaattttattcgcgtggatgaagaagcgtttctcgcctgcccggaagagtcggtggattacgcggtcatggaacgtacggcagatgctgttgtggtgccgatggatgcgggctggagcgatgttggctcctggtcttcattatgggagatcagcgcccacaccgccgagggcaacgtttgccacggcgatgtgattaatcacaaaactgaaaacagctatgtgtatgctgaatctggcctggtcaccaccgtcggggtgaaagatctggtagtggtgcagaccaaagatgcggtgctgattgccgaccgtaacgcggtacaggatgtgaaaaaagtggtcgagcagatcaaagccgatggtcgccatgagcatcgggtgcatcgcgaagtgtatcgtccgtggggcaaatatgactctatcgacgcgggcgaccgctaccaggtgaaacgcatcaccgtgaaaccgggcgagggcttgtcggtacagatgcaccatcaccgcgcggaacactgggtggttgtcgcgggaacggcaaaagtcaccattgatggtgatatcaaactgcttggtgaaaacgagtccatttatattccgctgggggcgacgcattgcctggaaaacccggggaaaattccgctcgatttaattgaagtgcgctccggctcttatctcgaagaggatgatgtggtgcgtttcgcggatcgctacggacgggtgtaaacgtcgcatcaggcaatgaatgcgaaaccgcggtgtaaataacgacaaaaataaaattggccgcttcggtcagggccaactattgcctgaaaaagggtaacgatatgaaaaaattaacctgctttaaagcctatgatattcgcgggaaattaggcgaagaactgaatgaagatatcgcctggcgcattggtcgcgcctatggcgaatttctcaaaccgaaaaccattgtgttaggcggtgatgtccgcctcaccagcgaaaccttaaaactggcgctggcgaaaggtttacaggatgcgggcgttgacgtgctggatattggtatgtccggcaccgaagagatctatttcgccacgttccatctcggcgtggatggcggcattgaagttaccgccagccataatccgatggattataacggcatgaagctggttcgcgagggggctcgcccgatcagcggagataccggactgcgcgacgtccagcgtctggctgaagccaacgactttcctcccgtcgatgaaaccaaacgcggtcgctatcagcaaatcaacctgcgtgacgcttacgttgatcacctgttcggttatatcaatgtcaaaaacctcacgccgctcaagctggtgatcaactccgggaacggcgcagcgggtccggtggtggacgccattgaagcccgctttaaagccctcggcgcgcccgtggaattaatcaaagtgcacaacacgccggacggcaatttccccaacggtattcctaacccactactgccggaatgccgcgacgacacccgcaatgcggtcatcaaacacggcgcggatatgggcattgcttttgatggcgattttgaccgctgtttcctgtttgacgaaaaagggcagtttattgagggctactacattgtcggcctgttggcagaagcattcctcgaaaaaaatcccggcgcgaagatcatccacgatccacgtctctcctggaacaccgttgatgtggtgactgccgcaggtggcacgccggtaatgtcgaaaaccggacacgcctttattaaagaacgtatgcgcaaggaagacgccatctatggtggcgaaatgagcgcccaccattacttccgtgatttcgcttactgcgacagcggcatgatcccgtggctgctggtcgccgaactggtgtgcctgaaagataaaacgctgggcgaactggtacgcgaccggatggcggcgtttccggcaagcggtgagatcaacagcaaactggcgcaacccgttgaggcgattaaccgcgtggaacagcattttagccgtgaggcgctggcggtggatcgcaccgatggcatcagcatgacctttgccgactggcgctttaacctgcgcacctccaataccgaaccggtggtgcgcctgaatgtggaatcgcgcggtgatgtgccgctgatggaagcgcgaacgcgaactctgctgacgttgctgaacgagtaa

SEQ ID NO: 12 - protein, 332 aa, Neisseria meningitidis
MQPLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRPEDIADFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTLPAGAWLDFAADGRMRRLFTLRQYFGILHRLLKNR

SEQ ID NO: 13 - protein, 439 aa, Helicobacter pylori
MISVYIISLKESQRRLDTEKLVLESNEKFKGRCVFQIFDAISPKHEDFEKFVQELYDSSSLLKSDWFHSDYCYQELLPQEFGCYLSHYLLWKECVKLNQPVVILEDDVALESNFMQALEDCLKSPFDFVRLYGHYWGGHKTNLCALPVYTETEEAEASIEKTPIENYEVTSPPPPNPTRDTQQDFITETQQDPKELSEPCKIAPQKISFNQVVFKKIKRKLNRFIGSILARTEVYKNIVAKYDDLTTKYDDLTTKYDDLTTKYDDLTTKYDDLNKNIAEKYDELMGKYESLLAKEVNIKETFWESRADSEKEALFLDHFYLTSVYVATTAGYYLTPKGAKTFIEATERFKIIEPVDMFINNPTYHDIANFTYVPCPVSLNKHAFNSTIQNAKKPDISLKPPKKSYFDNLFYHKFNARKCLKAFNKYSKQYAPLKTPKEV

SEQ ID NO: 14 - protein, 417 aa, Escherichia coli [sequence omitted]

SEQ ID NO: 15 - DNA, 203 bp, Synthetic construct
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaagagaaaaacagct

SEQ ID NO: 16 - DNA, 203 bp, Synthetic construct
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaactgagaaacagct

SEQ ID NO: 17 - DNA, 203 bp, Synthetic construct
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaacaaaaaccggagatacc

SEQ ID NO: 18 - DNA, 141 bp, Synthetic construct
  source 1..141: mol_type = other DNA
tgtgagttagctcactcattaggcaccccaggctttacactttatgcttccggctcgtatgttgtgtggaatgcctacaagcatcgtggaggtccgtgactttcacgcatacaacaaacattaaccaaggaggaaacagct

SEQ ID NO: 19 - DNA, 203 bp, Synthetic construct
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaaaggaaaaacagct

SEQ ID NO: 20 - DNA, 203 bp, Synthetic construct
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaactaggaaacagct

SEQ ID NO: 21 - DNA, 203 bp, Synthetic construct
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaaccgagaaacagct

SEQ ID NO: 22 - DNA, 310 bp, Synthetic construct
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaactaggaaacagct

SEQ ID NO: 23 - DNA, 203 bp, Synthetic construct
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaagagcaaaacagct

SEQ ID NO: 24 - DNA, 203 bp, Synthetic construct
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaaggaggaaacagct

SEQ ID NO: 25 - DNA, 189 bp, Synthetic construct
  source 1..189: mol_type = other DNA
gaaaacattcataaattaaatgtgaattgccgcacacattattaaataagatttacaaaatgttcaaaatgacgcatgaaatcacgtttcactttcgaattatgagcgaatatgcgcgatgcctacaagcatcgtggaggtccgtgactttcacgcatacaacaaacattaaccaaggaggaaacagct

SEQ ID NO: 26 - DNA, 239 bp, Synthetic construct
  source 1..239: mol_type = other DNA
ccatttagccatagtaaaaacatgaattgtttgatttcgcgcatattcgctcataattcgaaagtgaaacgtgatttcatgcgtcattttgaacattttgtaaatcttatttaataatgtgtgcggcaattcacatttaatttatgaatgttttcttaacatcgcggcatgcctacaagcatcgtggaggtccgtgactttcacgcatacaacaaacattaaccaaggaggaaacagct

SEQ ID NO: 27 - DNA, 300 bp, Escherichia coli
  source 1..300: mol_type = genomic DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaggaggaaacagct

SEQ ID NO: 28 - DNA, 310 bp, Synthetic construct
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaactgagaaacagct

SEQ ID NO: 29 - DNA, 310 bp, Synthetic construct
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaccgagaaacagct

SEQ ID NO: 30 - DNA, 310 bp, Synthetic construct
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagagaaaaacagct

SEQ ID NO: 31 - DNA, 350 bp, Synthetic construct
  source 1..350: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgttttaacgttgtaacccgtatgtaacagtgaataatcacttttgccgaggtaacagcgtcataacaacaattaaagccgttttctggagcgttaccgggcatggaagaacgaattttaaaaagtgagcttcggcgttcagtaacacttcattaactctactgccccgccgagcatttatctcaagcactaccctgcataagcaaggaggaaacagct

SEQ ID NO: 32 - DNA, 310 bp, Synthetic construct
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaaggaaaaacagct

SEQ ID NO: 33 - DNA, 300 bp, Synthetic construct
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagagcaaaacagct

SEQ ID NO: 34 - DNA, 310 bp, Synthetic construct
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagagctaaacagct

SEQ ID NO: 35 - DNA, 189 bp, Synthetic construct; identical to SEQ ID NO: 25
  source 1..189: mol_type = other DNA

SEQ ID NO: 36 - DNA, 107 bp, Escherichia coli
  source 1..107: mol_type = genomic DNA
tgtgagttagctcactcattaggcaccccaggctttacactttatgcttccggctcgtatgttgtgtggaattgtgagcggataacaatttcacacaggaaacagct

SEQ ID NO: 37 - DNA, 300 bp, Synthetic construct
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagaacaaaacagct

SEQ ID NO: 38 - DNA, 300 bp, Synthetic construct
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaattcgaaacagct

SEQ ID NO: 39 - protein, 432 aa, Helicobacter pylori
MFQPLLDAYIDSTRLDETDYKPPLKIAVANWWGGVEEFKKSTLYFILSQRYTITLHRNPDKPADIVFGNPLGSARKILSYQNAKRVFYTGENEVPNFNLFDYAIGFDELDFNDRYLRMPLYYAHLHYEAELVNDTTSPYKIKDNSLYALKKPSHHFKENHPNLCAVVNNESDPLKRGFASFVASNPNAPKRNAFYDALNSIEPVTGGGSVKNTLGYNVKNKNEFLSQYKFNLCFENSQGYGYVTEKILDAYFSHTIPIYWGSPSVAKDFNPKSFVNVHDFNNFDEAIDHVRYLHTHPNAYLDMLYENPLNTLDGKAYFYQNLSFKKILDFFKTILENDTIYHCDAHNYSALHRDLNEPLVSIDDLRINYDDLRINYDDLRINYDDLRINYERLLQNASPLLELSQNTSFKIYRKAYQKSLPLLRAIRRWVKK

SEQ ID NO: 40 - DNA, 1299 bp, synthetic construct
  source 1..1299: mol_type = other DNA
atgttccagccgctgctggatgcgtacattgacagcacccgtctggacgagaccgattataagccgccgctgaaaatcgcggtggcgaactggtggggtggcgttgaggaattcaagaaaagcaccctgtactttatcctgagccagcgttataccattaccctgcaccgtaacccggataaaccggcggacattgtgttcggcaacccgctgggtagcgcgcgtaaaatcctgagctaccaaaacgcgaagcgtgtgttttataccggcgagaacgaagttccgaacttcaacctgtttgattacgcgattggtttcgacgaactggattttaacgaccgttacctgcgtatgccgctgtactatgcgcacctgcactatgaggcggaactggttaacgataccaccagcccgtacaagatcaaagacaacagcctgtatgcgctgaagaaaccgagccaccacttcaaagagaaccacccgaacctgtgcgcggtggttaacaacgaaagcgatccgctgaaacgtggcttcgcgagctttgtggcgagcaacccgaacgcgccgaagcgtaacgcgttttacgacgcgctgaacagcattgagccggtgaccggtggcggtagcgttaagaacaccctgggttacaacgttaagaacaaaaacgaattcctgagccagtataaattcaacctgtgctttgagaacagccaaggctacggttatgtgaccgaaaagattctggacgcgtactttagccacaccatcccgatttattggggcagcccgagcgttgcgaaagatttcaacccgaagagctttgtgaacgttcacgacttcaacaactttgatgaggcgatcgaccacgtgcgttacctgcacacccacccgaacgcgtacctggatatgctgtatgaaaacccgctgaacaccctggacggtaaagcgtacttctatcagaacctgagcttcaagaaaattctggatttctttaagaccatcctggagaacgataccatttaccactgcgacgcgcacaactatagcgcgctgcaccgtgacctgaacgaaccgctggttagcatcgacgatctgcgtattaactacgacgatctgcgtatcaactatgatgatctgcgtatcaattatgacgatctgcgtattaattatgagcgtctgctgcagaacgcgagcccgctgctggaactgagccaaaacaccagcttcaagatttaccgtaaagcgtatcaaaagagcctgccgctgctgcgtgcgatccgtcgttgggtgaagaaataa